SEQUENCE LISTING (184 sequences)

SEQ ID NO: 1 - protein, 15 aa, synthetic construct
  REGION 1..15: note = Synthetic sequence
  REGION 1..15: note = MISC_FEATURE - peptide, FOL-005
VDTYDGDISVVYGLR

SEQ ID NO: 2 - protein, 15 aa, synthetic construct
  REGION 1..15: note = Synthetic sequence
  REGION 1..15: note = MISC_FEATURE - peptide
VDTYDGDISVVYGLS

SEQ ID NO: 3 - protein, 14 aa, synthetic construct
  REGION 1..14: note = Synthetic sequence
  REGION 1..14: note = MISC_FEATURE - peptide, FOL-025
VDTYDGDISVVYGL

SEQ ID NO: 4 - protein, 14 aa, synthetic construct
  REGION 1..14: note = Synthetic sequence
  REGION 1..14: note = MISC_FEATURE - peptide
DTYDGDISVVYGLR

SEQ ID NO: 5 - protein, 14 aa, synthetic construct
  REGION 1..14: note = Synthetic sequence
  REGION 1..14: note = MISC_FEATURE - peptide
TYDGDISVVYGLRS

SEQ ID NO: 6 - protein, 13 aa, synthetic construct
  REGION 1..13: note = Synthetic sequence
  REGION 1..13: note = MISC_FEATURE - peptide, FOL-024
VDTYDGDISVVYG

SEQ ID NO: 7 - protein, 13 aa, synthetic construct
  REGION 1..13: note = Synthetic sequence
  REGION 1..13: note = MISC_FEATURE - peptide
DTYDGDISVVYGL

SEQ ID NO: 8 - protein, 13 aa, synthetic construct
  REGION 1..13: note = Synthetic sequence
  REGION 1..13: note = MISC_FEATURE - peptide
TYDGDISVVYGLR

SEQ ID NO: 9 - protein, 13 aa, synthetic construct
  REGION 1..13: note = Synthetic sequence
  REGION 1..13: note = MISC_FEATURE - peptide
YDGDISVVYGLRS

SEQ ID NO: 10 - protein, 12 aa, synthetic construct
  REGION 1..12: note = Synthetic sequence
  REGION 1..12: note = MISC_FEATURE - peptide
VDTYDGDISVVY

SEQ ID NO: 11 - protein, 12 aa, synthetic construct
  REGION 1..12: note = Synthetic sequence
  REGION 1..12: note = MISC_FEATURE - peptide
DTYDGDISVVYG

SEQ ID NO: 12 - protein, 12 aa, synthetic construct
  REGION 1..12: note = Synthetic sequence
  REGION 1..12: note = MISC_FEATURE - peptide
TYDGDISVVYGL

SEQ ID NO: 13 - protein, 12 aa, synthetic construct
  REGION 1..12: note = Synthetic sequence
  REGION 1..12: note = MISC_FEATURE - peptide
YDGDISVVYGLR

SEQ ID NO: 14 - protein, 12 aa, synthetic construct
  REGION 1..12: note = Synthetic sequence
  REGION 1..12: note = MISC_FEATURE - peptide
DGDISVVYGLRS

SEQ ID NO: 15 - protein, 11 aa, synthetic construct
  REGION 1..11: note = Synthetic sequence
  REGION 1..11: note = MISC_FEATURE - peptide
VDTYDGDISVV

SEQ ID NO: 16 - protein, 11 aa, synthetic construct
  REGION 1..11: note = Synthetic sequence
  REGION 1..11: note = MISC_FEATURE - peptide
DTYDGDISVVY

SEQ ID NO: 17 - protein, 11 aa, synthetic construct
  REGION 1..11: note = Synthetic sequence
  REGION 1..11: note = MISC_FEATURE - peptide
TYDGDISVVYG

SEQ ID NO: 18 - protein, 11 aa, synthetic construct
  REGION 1..11: note = Synthetic sequence
  REGION 1..11: note = MISC_FEATURE - peptide
YDGDISVVYGL

SEQ ID NO: 19 - protein, 11 aa, synthetic construct
  REGION 1..11: note = Synthetic sequence
  REGION 1..11: note = MISC_FEATURE - peptide
DGDISVVYGLR

SEQ ID NO: 20 - protein, 11 aa, synthetic construct
  REGION 1..11: note = Synthetic sequence
  REGION 1..11: note = MISC_FEATURE - peptide
GDISVVYGLRS

SEQ ID NO: 21 - protein, 10 aa, synthetic construct
  REGION 1..10: note = Synthetic sequence
  REGION 1..10: note = MISC_FEATURE - peptide
VDTYDGDISV

SEQ ID NO: 22 - protein, 10 aa, synthetic construct
  REGION 1..10: note = Synthetic sequence
  REGION 1..10: note = MISC_FEATURE - peptide
DTYDGDISVV

SEQ ID NO: 23 - protein, 10 aa, synthetic construct
  REGION 1..10: note = Synthetic sequence
  REGION 1..10: note = MISC_FEATURE - peptide
TYDGDISVVY

SEQ ID NO: 24 - protein, 10 aa, synthetic construct
  REGION 1..10: note = Synthetic sequence
  REGION 1..10: note = MISC_FEATURE - peptide
YDGDISVVYG

SEQ ID NO: 25 - protein, 10 aa, synthetic construct
  REGION 1..10: note = Synthetic sequence
  REGION 1..10: note = MISC_FEATURE - peptide
DGDISVVYGL

SEQ ID NO: 26 - protein, 10 aa, synthetic construct
  REGION 1..10: note = Synthetic sequence
  REGION 1..10: note = MISC_FEATURE - peptide, FOL-009h
GDISVVYGLR

SEQ ID NO: 27 - protein, 10 aa, synthetic construct
  REGION 1..10: note = Synthetic sequence
  REGION 1..10: note = MISC_FEATURE - peptide
DISVVYGLRS

SEQ ID NO: 28 - protein, 9 aa, synthetic construct
  REGION 1..9: note = Synthetic sequence
  REGION 1..9: note = MISC_FEATURE - peptide, FOL-019h
VDTYDGDIS

SEQ ID NO: 29 - protein, 9 aa, synthetic construct
  REGION 1..9: note = Synthetic sequence
  REGION 1..9: note = MISC_FEATURE - peptide
DTYDGDISV

SEQ ID NO: 30 - protein, 9 aa, synthetic construct
  REGION 1..9: note = Synthetic sequence
  REGION 1..9: note = MISC_FEATURE - peptide
TYDGDISVV

SEQ ID NO: 31 - protein, 9 aa, synthetic construct
  REGION 1..9: note = Synthetic sequence
  REGION 1..9: note = MISC_FEATURE - peptide
YDGDISVVY

SEQ ID NO: 32 - protein, 9 aa, synthetic construct
  REGION 1..9: note = Synthetic sequence
  REGION 1..9: note = MISC_FEATURE - peptide
DGDISVVYG

SEQ ID NO: 33 - protein, 9 aa, synthetic construct
  REGION 1..9: note = Synthetic sequence
  REGION 1..9: note = MISC_FEATURE - peptide
GDISVVYGL

SEQ ID NO: 34 - protein, 9 aa, synthetic construct
  REGION 1..9: note = Synthetic sequence
  REGION 1..9: note = MISC_FEATURE - peptide
DISVVYGLR

SEQ ID NO: 35 - protein, 9 aa, synthetic construct
  REGION 1..9: note = Synthetic sequence
  REGION 1..9: note = MISC_FEATURE - peptide
ISVVYGLRS

SEQ ID NO: 36 - protein, 8 aa, synthetic construct
  REGION 1..8: note = Synthetic sequence
  REGION 1..8: note = MISC_FEATURE - peptide
VDTYDGDI

SEQ ID NO: 37 - protein, 8 aa, synthetic construct
  REGION 1..8: note = Synthetic sequence
  REGION 1..8: note = MISC_FEATURE - peptide
DTYDGDIS

SEQ ID NO: 38 - protein, 8 aa, synthetic construct
  REGION 1..8: note = Synthetic sequence
  REGION 1..8: note = MISC_FEATURE - peptide
TYDGDISV

SEQ ID NO: 39 - protein, 8 aa, synthetic construct
  REGION 1..8: note = Synthetic sequence
  REGION 1..8: note = MISC_FEATURE - peptide
YDGDISVV

SEQ ID NO: 40 - protein, 8 aa, synthetic construct
  REGION 1..8: note = Synthetic sequence
  REGION 1..8: note = MISC_FEATURE - peptide
DGDISVVY

SEQ ID NO: 41 - protein, 8 aa, synthetic construct
  REGION 1..8: note = Synthetic sequence
  REGION 1..8: note = MISC_FEATURE - peptide
GDISVVYG

SEQ ID NO: 42 - protein, 8 aa, synthetic construct
  REGION 1..8: note = Synthetic sequence
  REGION 1..8: note = MISC_FEATURE - peptide
DISVVYGL

SEQ ID NO: 43 - protein, 8 aa, synthetic construct
  REGION 1..8: note = Synthetic sequence
  REGION 1..8: note = MISC_FEATURE - peptide
ISVVYGLR

SEQ ID NO: 44 - protein, 7 aa, synthetic construct
  REGION 1..7: note = Synthetic sequence
  REGION 1..7: note = MISC_FEATURE - peptide
VDTYDGD

SEQ ID NO: 45 - protein, 7 aa, synthetic construct
  REGION 1..7: note = Synthetic sequence
  REGION 1..7: note = MISC_FEATURE - peptide
DTYDGDI

SEQ ID NO: 46 - protein, 7 aa, synthetic construct
  REGION 1..7: note = Synthetic sequence
  REGION 1..7: note = MISC_FEATURE - peptide
TYDGDIS

SEQ ID NO: 47 - protein, 7 aa, synthetic construct
  REGION 1..7: note = Synthetic sequence
  REGION 1..7: note = MISC_FEATURE - peptide
YDGDISV

SEQ ID NO: 48 - protein, 7 aa, synthetic construct
  REGION 1..7: note = Synthetic sequence
  REGION 1..7: note = MISC_FEATURE - peptide
DGDISVV

SEQ ID NO: 49 - protein, 7 aa, synthetic construct
  REGION 1..7: note = Synthetic sequence
  REGION 1..7: note = MISC_FEATURE - peptide
GDISVVY

SEQ ID NO: 50 - protein, 7 aa, synthetic construct
  REGION 1..7: note = Synthetic sequence
  REGION 1..7: note = MISC_FEATURE - peptide
DISVVYG

SEQ ID NO: 51 - protein, 7 aa, synthetic construct
  REGION 1..7: note = Synthetic sequence
  REGION 1..7: note = MISC_FEATURE - peptide
ISVVYGL

SEQ ID NO: 52 - protein, 6 aa, synthetic construct
  REGION 1..6: note = Synthetic sequence
  REGION 1..6: note = MISC_FEATURE - peptide
DTYDGD

SEQ ID NO: 53 - protein, 6 aa, synthetic construct
  REGION 1..6: note = Synthetic sequence
  REGION 1..6: note = MISC_FEATURE - peptide
TYDGDI

SEQ ID NO: 54 - protein, 6 aa, synthetic construct
  REGION 1..6: note = Synthetic sequence
  REGION 1..6: note = MISC_FEATURE - peptide
YDGDIS

SEQ ID NO: 55 - protein, 6 aa, synthetic construct
  REGION 1..6: note = Synthetic sequence
  REGION 1..6: note = MISC_FEATURE - peptide
DGDISV

SEQ ID NO: 56 - protein, 6 aa, synthetic construct
  REGION 1..6: note = Synthetic sequence
  REGION 1..6: note = MISC_FEATURE - peptide
GDISVV

SEQ ID NO: 57 - protein, 6 aa, synthetic construct
  REGION 1..6: note = Synthetic sequence
  REGION 1..6: note = MISC_FEATURE - peptide
DISVVY

SEQ ID NO: 58 - protein, 6 aa, synthetic construct
  REGION 1..6: note = Synthetic sequence
  REGION 1..6: note = MISC_FEATURE - peptide
ISVVYG

SEQ ID NO: 59 - protein, 5 aa, synthetic construct
  REGION 1..5: note = Synthetic sequence
  REGION 1..5: note = MISC_FEATURE - peptide
TYDGD

SEQ ID NO: 60 - protein, 5 aa, synthetic construct
  REGION 1..5: note = Synthetic sequence
  REGION 1..5: note = MISC_FEATURE - peptide
YDGDI

SEQ ID NO: 61 - protein, 5 aa, synthetic construct
  REGION 1..5: note = Synthetic sequence
  REGION 1..5: note = MISC_FEATURE - peptide
DGDIS

SEQ ID NO: 62 - protein, 5 aa, synthetic construct
  REGION 1..5: note = Synthetic sequence
  REGION 1..5: note = MISC_FEATURE - peptide
GDISV

SEQ ID NO: 63 - protein, 5 aa, synthetic construct
  REGION 1..5: note = Synthetic sequence
  REGION 1..5: note = MISC_FEATURE - peptide
DISVV

SEQ ID NO: 64 - protein, 5 aa, synthetic construct
  REGION 1..5: note = Synthetic sequence
  REGION 1..5: note = MISC_FEATURE - peptide
ISVVY

SEQ ID NO: 65 - protein, 5 aa, synthetic construct
  REGION 1..5: note = Synthetic sequence
  REGION 1..5: note = MISC_FEATURE - peptide
SVVYG

SEQ ID NO: 66 - protein, 300 aa, Homo sapiens
  REGION 1..300: note = MISC_FEATURE - Wildtype human osteopontin
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 67 - protein, 16 aa, synthetic construct
  REGION 1..16: note = Synthetic sequence
  REGION 1..16: note = MISC_FEATURE - peptide, FOL-002
VDTYDGRGDSVVYGLR

SEQ ID NO: 68 - protein, 15 aa, synthetic construct
  REGION 1..15: note = Synthetic sequence
  REGION 1..15: note = MISC_FEATURE - peptide
  SITE 3: note = MISC_FEATURE - Z is T or V
  SITE 4: note = MISC_FEATURE - Z is Y or P
  SITE 5: note = MISC_FEATURE - Z is D or N
  SITE 7: note = MISC_FEATURE - Z is D or G
  SITE 8: note = MISC_FEATURE - Z is I or G
  SITE 10: note = MISC_FEATURE - Z is V or L
  SITE 11: note = MISC_FEATURE - Z is V or A
VDZZZGZZSZZYGLR

SEQ ID NO: 69 - protein, 15 aa, synthetic construct
  REGION 1..15: note = Synthetic sequence
  REGION 1..15: note = MISC_FEATURE - peptide, FOL-004
VDVPNGDISLAYGLR

SEQ ID NO: 70 - protein, 15 aa, synthetic construct
  REGION 1..15: note = Synthetic sequence
  REGION 1..15: note = MISC_FEATURE - peptide
DVPNGDISLAYGLRS

SEQ ID NO: 71 - protein, 14 aa, synthetic construct
  REGION 1..14: note = Synthetic sequence
  REGION 1..14: note = MISC_FEATURE - peptide
VDVPNGDISLAYGL

SEQ ID NO: 72 - protein, 14 aa, synthetic construct
  REGION 1..14: note = Synthetic sequence
  REGION 1..14: note = MISC_FEATURE - peptide
DVPNGDISLAYGLR

SEQ ID NO: 73 - protein, 14 aa, synthetic construct
  REGION 1..14: note = Synthetic sequence
  REGION 1..14: note = MISC_FEATURE - peptide
VPNGDISLAYGLRS

SEQ ID NO: 74 - protein, 13 aa, synthetic construct
  REGION 1..13: note = Synthetic sequence
  REGION 1..13: note = MISC_FEATURE - peptide, FOL-017
VDVPNGDISLAYG

SEQ ID NO: 75 - protein, 13 aa, synthetic construct
  REGION 1..13: note = Synthetic sequence
  REGION 1..13: note = MISC_FEATURE - peptide
DVPNGDISLAYGL

SEQ ID NO: 76 - protein, 13 aa, synthetic construct
  REGION 1..13: note = Synthetic sequence
  REGION 1..13: note = MISC_FEATURE - peptide
VPNGDISLAYGLR

SEQ ID NO: 77 - protein, 13 aa, synthetic construct
  REGION 1..13: note = Synthetic sequence
  REGION 1..13: note = MISC_FEATURE - peptide
PNGDISLAYGLRS

SEQ ID NO: 78 - protein, 12 aa, synthetic construct
  REGION 1..12: note = Synthetic sequence
  REGION 1..12: note = MISC_FEATURE - peptide
VDVPNGDISLAY

SEQ ID NO: 79 - protein, 12 aa, synthetic construct
  REGION 1..12: note = Synthetic sequence
  REGION 1..12: note = MISC_FEATURE - peptide
DVPNGDISLAYG

SEQ ID NO: 80 - protein, 12 aa, synthetic construct
  REGION 1..12: note = Synthetic sequence
  REGION 1..12: note = MISC_FEATURE - peptide
VPNGDISLAYGL

SEQ ID NO: 81 - protein, 12 aa, synthetic construct
  REGION 1..12: note = Synthetic sequence
  REGION 1..12: note = MISC_FEATURE - peptide
PNGDISLAYGLR

SEQ ID NO: 82 - protein, 12 aa, synthetic construct
  REGION 1..12: note = Synthetic sequence
  REGION 1..12: note = MISC_FEATURE - peptide
NGDISLAYGLRS

SEQ ID NO: 83 - protein, 11 aa, synthetic construct
  REGION 1..11: note = Synthetic sequence
  REGION 1..11: note = MISC_FEATURE - peptide
VDVPNGDISLA

SEQ ID NO: 84 - protein, 11 aa, synthetic construct
  REGION 1..11: note = Synthetic sequence
  REGION 1..11: note = MISC_FEATURE - peptide
DVPNGDISLAY

SEQ ID NO: 85 - protein, 11 aa, synthetic construct
  REGION 1..11: note = Synthetic sequence
  REGION 1..11: note = MISC_FEATURE - peptide
VPNGDISLAYG

SEQ ID NO: 86 - protein, 11 aa, synthetic construct
  REGION 1..11: note = Synthetic sequence
  REGION 1..11: note = MISC_FEATURE - peptide
PNGDISLAYGL

SEQ ID NO: 87 - protein, 11 aa, synthetic construct
  REGION 1..11: note = Synthetic sequence
  REGION 1..11: note = MISC_FEATURE - peptide
NGDISLAYGLR

SEQ ID NO: 88 - protein, 11 aa, synthetic construct
  REGION 1..11: note = Synthetic sequence
  REGION 1..11: note = MISC_FEATURE - peptide
GDISLAYGLRS

SEQ ID NO: 89 - protein, 10 aa, synthetic construct
  REGION 1..10: note = Synthetic sequence
  REGION 1..10: note = MISC_FEATURE - peptide
VDVPNGDISL

SEQ ID NO: 90 - protein, 10 aa, synthetic construct
  REGION 1..10: note = Synthetic sequence
  REGION 1..10: note = MISC_FEATURE - peptide
DVPNGDISLA

SEQ ID NO: 91 - protein, 10 aa, synthetic construct
  REGION 1..10: note = Synthetic sequence
  REGION 1..10: note = MISC_FEATURE - peptide
VPNGDISLAY

SEQ ID NO: 92 - protein, 10 aa, synthetic construct
  REGION 1..10: note = Synthetic sequence
  REGION 1..10: note = MISC_FEATURE - peptide
PNGDISLAYG

SEQ ID NO: 93 - protein, 10 aa, synthetic construct
  REGION 1..10: note = Synthetic sequence
  REGION 1..10: note = MISC_FEATURE - peptide
NGDISLAYGL

SEQ ID NO: 94 - protein, 10 aa, synthetic construct
  REGION 1..10: note = Synthetic sequence
  REGION 1..10: note = MISC_FEATURE - peptide, FOL-009
GDISLAYGLR

SEQ ID NO: 95 - protein, 10 aa, synthetic construct
  REGION 1..10: note = Synthetic sequence
  REGION 1..10: note = MISC_FEATURE - peptide
DISLAYGLRS

SEQ ID NO: 96 - protein, 9 aa, synthetic construct
  REGION 1..9: note = Synthetic sequence
  REGION 1..9: note = MISC_FEATURE - peptide
  REGION 1..9: note = MISC_FEATURE - peptide, FOL-019
VDVPNGDIS

SEQ ID NO: 97 - protein, 9 aa, synthetic construct
  REGION 1..9: note = Synthetic sequence
  REGION 1..9: note = MISC_FEATURE - peptide
DVPNGDISL

SEQ ID NO: 98 - protein, 9 aa, synthetic construct
  REGION 1..9: note = Synthetic sequence
  REGION 1..9: note = MISC_FEATURE - peptide
VPNGDISLA

SEQ ID NO: 99 - protein, 9 aa, synthetic construct
  REGION 1..9: note = Synthetic sequence
  REGION 1..9: note = MISC_FEATURE - peptide
PNGDISLAY

SEQ ID NO: 100 - protein, 9 aa, synthetic construct
  REGION 1..9: note = Synthetic sequence
  REGION 1..9: note = MISC_FEATURE - peptide
NGDISLAYG

SEQ ID NO: 101 - protein, 9 aa, synthetic construct
  REGION 1..9: note = Synthetic sequence
  REGION 1..9: note = MISC_FEATURE - peptide
GDISLAYGL

SEQ ID NO: 102 - protein, 9 aa, synthetic construct
  REGION 1..9: note = Synthetic sequence
  REGION 1..9: note = MISC_FEATURE - peptide
DISLAYGLR

SEQ ID NO: 103 - protein, 9 aa, synthetic construct
  REGION 1..9: note = Synthetic sequence
  REGION 1..9: note = MISC_FEATURE - peptide
ISLAYGLRS

SEQ ID NO: 104 - protein, 8 aa, synthetic construct
  REGION 1..8: note = Synthetic sequence
  REGION 1..8: note = MISC_FEATURE - peptide
VDVPNGDI

SEQ ID NO: 105 - protein, 8 aa, synthetic construct
  REGION 1..8: note = Synthetic sequence
  REGION 1..8: note = MISC_FEATURE - peptide
DVPNGDIS

SEQ ID NO: 106 - protein, 8 aa, synthetic construct
  REGION 1..8: note = Synthetic sequence
  REGION 1..8: note = MISC_FEATURE - peptide
VPNGDISL

SEQ ID NO: 107 - protein, 8 aa, synthetic construct
  REGION 1..8: note = Synthetic sequence
  REGION 1..8: note = MISC_FEATURE - peptide
PNGDISLA

SEQ ID NO: 108 - protein, 8 aa, synthetic construct
  REGION 1..8: note = Synthetic sequence
  REGION 1..8: note = MISC_FEATURE - peptide
NGDISLAY

SEQ ID NO: 109 - protein, 8 aa, synthetic construct
  REGION 1..8: note = Synthetic sequence
  REGION 1..8: note = MISC_FEATURE - peptide
GDISLAYG

SEQ ID NO: 110 - protein, 8 aa, synthetic construct
  REGION 1..8: note = Synthetic sequence
  REGION 1..8: note = MISC_FEATURE - peptide
DISLAYGL

SEQ ID NO: 111 - protein, 8 aa, synthetic construct
  REGION 1..8: note = Synthetic sequence
  REGION 1..8: note = MISC_FEATURE - peptide
ISLAYGLR

SEQ ID NO: 112 - protein, 7 aa, synthetic construct
  REGION 1..7: note = Synthetic sequence
  REGION 1..7: note = MISC_FEATURE - peptide
VDVPNGD

SEQ ID NO: 113 - protein, 7 aa, synthetic construct
  REGION 1..7: note = Synthetic sequence
  REGION 1..7: note = MISC_FEATURE - peptide
DVPNGDI

SEQ ID NO: 114 - protein, 7 aa, synthetic construct
  REGION 1..7: note = Synthetic sequence
  REGION 1..7: note = MISC_FEATURE - peptide
VPNGDIS

SEQ ID NO: 115 - protein, 7 aa, synthetic construct
  REGION 1..7: note = Synthetic sequence
  REGION 1..7: note = MISC_FEATURE - peptide
PNGDISL

SEQ ID NO: 116 - protein, 7 aa, synthetic construct
  REGION 1..7: note = Synthetic sequence
  REGION 1..7: note = MISC_FEATURE - peptide
NGDISLA

SEQ ID NO: 117 - protein, 7 aa, synthetic construct
  REGION 1..7: note = Synthetic sequence
  REGION 1..7: note = MISC_FEATURE - peptide
GDISLAY

SEQ ID NO: 118 - protein, 7 aa, synthetic construct
  REGION 1..7: note = Synthetic sequence
  REGION 1..7: note = MISC_FEATURE - peptide
DISLAYG

SEQ ID NO: 119 - protein, 7 aa, synthetic construct
  REGION 1..7: note = Synthetic sequence
  REGION 1..7: note = MISC_FEATURE - peptide
ISLAYGL

SEQ ID NO: 120 - protein, 6 aa, synthetic construct
  REGION 1..6: note = Synthetic sequence
  REGION 1..6: note = MISC_FEATURE - peptide
DVPNGD

SEQ ID NO: 121 - protein, 6 aa, synthetic construct
  REGION 1..6: note = Synthetic sequence
  REGION 1..6: note = MISC_FEATURE - peptide
VPNGDI

SEQ ID NO: 122 - protein, 6 aa, synthetic construct
  REGION 1..6: note = Synthetic sequence
  REGION 1..6: note = MISC_FEATURE - peptide
PNGDIS

SEQ ID NO: 123 - protein, 6 aa, synthetic construct
  REGION 1..6: note = Synthetic sequence
  REGION 1..6: note = MISC_FEATURE - peptide
NGDISL

SEQ ID NO: 124 - protein, 6 aa, synthetic construct
  REGION 1..6: note = Synthetic sequence
  REGION 1..6: note = MISC_FEATURE - peptide
GDISLA

SEQ ID NO: 125 - protein, 6 aa, synthetic construct
  REGION 1..6: note = Synthetic sequence
  REGION 1..6: note = MISC_FEATURE - peptide
DISLAY

SEQ ID NO: 126 - protein, 6 aa, synthetic construct
  REGION 1..6: note = Synthetic sequence
  REGION 1..6: note = MISC_FEATURE - peptide
ISLAYG

SEQ ID NO: 127 - protein, 5 aa, synthetic construct
  REGION 1..5: note = Synthetic sequence
  REGION 1..5: note = MISC_FEATURE - peptide
VPNGD

SEQ ID NO: 128 - protein, 5 aa, synthetic construct
  REGION 1..5: note = Synthetic sequence
  REGION 1..5: note = MISC_FEATURE - peptide
PNGDI

SEQ ID NO: 129 - protein, 5 aa, synthetic construct
  REGION 1..5: note = Synthetic sequence
  REGION 1..5: note = MISC_FEATURE - peptide
NGDIS

SEQ ID NO: 130 - protein, 5 aa, synthetic construct
  REGION 1..5: note = Synthetic sequence
  REGION 1..5: note = MISC_FEATURE - peptide
GDISL

SEQ ID NO: 131 - protein, 5 aa, synthetic construct
  REGION 1..5: note = Synthetic sequence
  REGION 1..5: note = MISC_FEATURE - peptide
DISLA

SEQ ID NO: 132 - protein, 5 aa, synthetic construct
  REGION 1..5: note = Synthetic sequence
  REGION 1..5: note = MISC_FEATURE - peptide
ISLAY

SEQ ID NO: 133 - protein, 5 aa, synthetic construct
  REGION 1..5: note = Synthetic sequence
  REGION 1..5: note = MISC_FEATURE - peptide
SLAYG

SEQ ID NO: 134 - protein, 294 aa, Mus musculus
  REGION 1..294: note = MISC_FEATURE - Wildtype murine osteopontin
MRLAVICFCLFGIASSLPVKVTDSGSSEEKLYSLHPDPIATWLVPDPSQKQNLLAPQNAVSSEEKDDFKQETLPSNSNESHDHMDDDDDDDDDDGDHAESEDSVDSDESDESHHSDESDETVTASTQADTFTPIVPTVDVPNGRGDSLAYGLRSKSRSFQVSDEQYPDATDEDLTSHMKSGESKESLDVIPVAQLLSMPSDQDNNGKGSHESSQLDEPSLETHRLEHSKESQESADQSDVIDSQASSKASLEHQSHKFHSHKDKLVLDPKSKEDDRYLKFRISHELESSSSEVN

SEQ ID NO: 135 - protein, 16 aa, synthetic construct
  REGION 1..16: note = Synthetic sequence
  REGION 1..16: note = MISC_FEATURE - peptide, FOL-001
VDVPNGRGDSLAYGLR

SEQ ID NO: 136 - protein, 16 aa, synthetic construct
  REGION 1..16: note = Synthetic sequence
  REGION 1..16: note = MISC_FEATURE - peptide, FOL-014
KPLAEIDSIELSYGIK

SEQ ID NO: 137 - protein, 16 aa, synthetic construct
  REGION 1..16: note = Synthetic sequence
  REGION 1..16: note = MISC_FEATURE - peptide, FOL-003
GDPNDGRGDSVVYGLR

SEQ ID NO: 138 - protein, 15 aa, synthetic construct
  REGION 1..15: note = Synthetic sequence
  REGION 1..15: note = MISC_FEATURE - peptide, FOL-026
VDTYDGGISVVYGLR

SEQ ID NO: 139 - protein, 15 aa, synthetic construct
  REGION 1..15: note = Synthetic sequence
  REGION 1..15: note = MISC_FEATURE - peptide, FOL-027
VDTYDGDGSVVYGLR

SEQ ID NO: 140 - protein, 16 aa, synthetic construct
  REGION 1..16: note = Synthetic sequence
  REGION 1..16: note = MISC_FEATURE - peptide
  SITE 2: note = MISC_FEATURE - X is C, P or G
  SITE 5: note = MISC_FEATURE - X is E or G
  SITE 6: note = MISC_FEATURE - X is C, D or I
  SITE 7: note = MISC_FEATURE - X is D, I, S or G
  SITE 8: note = MISC_FEATURE - X is S, D or G
  SITE 10: note = MISC_FEATURE - X is E or G
  SITE 12: note = MISC_FEATURE - X is S or T
KXLAXXXXIXLXYGIK

SEQ ID NO: 141 - protein, 16 aa, synthetic construct
  REGION 1..16: note = Synthetic sequence
  REGION 1..16: note = MISC_FEATURE - peptide
KCLAECDSIELSYGIK

SEQ ID NO: 142 - protein, 8 aa, synthetic construct
  REGION 1..8: note = Synthetic sequence
  REGION 1..8: note = MISC_FEATURE - peptide
CLAEIDSC

SEQ ID NO: 143 - protein, 18 aa, synthetic construct
  REGION 1..18: note = Synthetic sequence
  REGION 1..18: note = MISC_FEATURE - peptide
CFKPLAEIDSIECSYGIK

SEQ ID NO: 144 - protein, 16 aa, synthetic construct
  REGION 1..16: note = Synthetic sequence
  REGION 1..16: note = MISC_FEATURE - peptide
KPLAEDISIELSYGIK

SEQ ID NO: 145 - protein, 16 aa, synthetic construct
  REGION 1..16: note = Synthetic sequence
  REGION 1..16: note = MISC_FEATURE - peptide
KPLAEISDIELSYGIK

SEQ ID NO: 146 - protein, 16 aa, synthetic construct
  REGION 1..16: note = Synthetic sequence
  REGION 1..16: note = MISC_FEATURE - peptide
KPLAEIGDIELSYGIK

SEQ ID NO: 147 - protein, 15 aa, synthetic construct
  REGION 1..15: note = Synthetic sequence
  REGION 1..15: note = MISC_FEATURE - peptide
KPLAEGDIELSYGIK

SEQ ID NO: 148 - protein, 13 aa, synthetic construct
  REGION 1..13: note = Synthetic sequence
  REGION 1..13: note = MISC_FEATURE - peptide
KPLAEIELSYGIK

SEQ ID NO: 149 - protein, 16 aa, synthetic construct
  REGION 1..16: note = Synthetic sequence
  REGION 1..16: note = MISC_FEATURE - peptide
KPLAEIDSIELTYGIK

SEQ ID NO: 150 - protein, 16 aa, synthetic construct
  REGION 1..16: note = Synthetic sequence
  REGION 1..16: note = MISC_FEATURE - peptide
KPLAEIDGIELSYGIK

SEQ ID NO: 151 - protein, 16 aa, synthetic construct
  REGION 1..16: note = Synthetic sequence
  REGION 1..16: note = MISC_FEATURE - peptide
KPLAEIDGIELTYGIK

SEQ ID NO: 152 - protein, 16 aa, synthetic construct
  REGION 1..16: note = Synthetic sequence
  REGION 1..16: note = MISC_FEATURE - peptide
KPLAEIGSIELSYGIK

SEQ ID NO: 153 - protein, 16 aa, synthetic construct
  REGION 1..16: note = Synthetic sequence
  REGION 1..16: note = MISC_FEATURE - peptide
KGLAEIDSIELSYGIK

SEQ ID NO: 154 - protein, 16 aa, synthetic construct
  REGION 1..16: note = Synthetic sequence
  REGION 1..16: note = MISC_FEATURE - peptide
KPLAGIDSIGLSYGIK

SEQ ID NO: 155 - protein, 16 aa, synthetic construct
  REGION 1..16: note = Synthetic sequence
  REGION 1..16: note = MISC_FEATURE - peptide
KCLAEIDSCELSYGIK

SEQ ID NO: 156 - protein, 13 aa, synthetic construct
  REGION 1..13: note = Synthetic sequence
  REGION 1..13: note = MISC_FEATURE - peptide
CFKPLAEIDSIEC

SEQ ID NO: 157 - protein, 15 aa, synthetic construct
  REGION 1..15: note = Synthetic sequence
  REGION 1..15: note = MISC_FEATURE - peptide
VDVPEGDISLAYGLR

SEQ ID NO: 158 - protein, 15 aa, synthetic construct
  REGION 1..15: note = Synthetic sequence
  REGION 1..15: note = MISC_FEATURE - peptide
LDGLVRAYDNISPVG

SEQ ID NO: 159 - protein, 14 aa, synthetic construct
  REGION 1..14: note = Synthetic sequence
  REGION 1..14: note = MISC_FEATURE - peptide
GDPNGDISVVYGLR

SEQ ID NO: 160 - protein, 15 aa, synthetic construct
  REGION 1..15: note = Synthetic sequence
  REGION 1..15: note = MISC_FEATURE - peptide
VDVPNGDISLAYRLR

SEQ ID NO: 161 - protein, 15 aa, synthetic construct
  REGION 1..15: note = Synthetic sequence
  REGION 1..15: note = MISC_FEATURE - peptide
VDVPEGDISLAYRLR

SEQ ID NO: 162 - protein, 16 aa, synthetic construct
  REGION 1..16: note = synthetic peptide
  REGION 1..16: note = MISC_FEATURE - peptide
  SITE 2: note = MISC_FEATURE - X is C, P or G
  SITE 5: note = MISC_FEATURE - X is E or G
  SITE 6: note = MISC_FEATURE - X is C, I or absent
  SITE 7: note = MISC_FEATURE - X is D, G or absent
  SITE 8: note = MISC_FEATURE - X is S, G or absent
  SITE 10: note = MISC_FEATURE - X is E or G
KXLAXXXXIXLSYGIK

SEQ ID NO: 163 - protein, 13 aa, synthetic construct
  REGION 1..13: note = synthetic peptide
  REGION 1..13: note = MISC_FEATURE - peptide
  SITE 2: note = MISC_FEATURE - X is C, P or G
  SITE 5: note = MISC_FEATURE - X is E or G
  SITE 10: note = MISC_FEATURE - X is E or G
KXLAXIXLSYGIK

SEQ ID NO: 164 - protein, 15 aa, synthetic construct
  REGION 1..15: note = synthetic peptide
  REGION 1..15: note = MISC_FEATURE - peptide
  REGION 1..5: note = MISC_FEATURE - Z is E or N
  SITE 5: note = MISC_FEATURE - Z is E or N
  SITE 13: note = MISC_FEATURE - Z is R or G
VDVPZGDISLAYZLR

SEQ ID NO: 165 - protein, 15 aa, synthetic construct
  REGION 1..15: note = synthetic peptide
  REGION 1..15: note = MISC_FEATURE - peptide
  SITE 7: note = MISC_FEATURE - Z is D or G
  SITE 8: note = MISC_FEATURE - Z is I or G
VDTYDGZZSVVYGLR

SEQ ID NO: 166 - protein, 16 aa, synthetic construct
  REGION 1..16: note = synthetic peptide
  REGION 1..16: note = MISC_FEATURE - peptide
  SITE 5: note = MISC_FEATURE - Z is D or G
  SITE 6: note = MISC_FEATURE - Z is D or G
  SITE 7: note = MISC_FEATURE - Z is I or R
  SITE 8: note = MISC_FEATURE - Z is G or absent
  SITE 9: note = MISC_FEATURE - Z is D or absent
GDPNZZZZZSVVYGLR

SEQ ID NO: 167 - protein, 15 aa, synthetic construct
  REGION 1..15: note = synthetic peptide
  REGION 1..15: note = MISC_FEATURE - peptide
  SITE 2: note = MISC_FEATURE - Z is beta D
VZTYDGDISVVYGLR

SEQ ID NO: 168 - protein, 15 aa, synthetic construct
  REGION 1..15: note = synthetic peptide
  REGION 1..15: note = MISC_FEATURE - peptide
  SITE 5: note = MISC_FEATURE - Z is beta D
VDTYZGDISVVYGLR

SEQ ID NO: 169 - protein, 15 aa, synthetic construct
  REGION 1..15: note = synthetic peptide
  REGION 1..15: note = MISC_FEATURE - peptide
  SITE 7: note = MISC_FEATURE - Z is beta D
VDTYDGZISVVYGLR

SEQ ID NO: 170 - protein, 14 aa, synthetic construct
  REGION 1..14: note = synthetic peptide
  REGION 1..14: note = MISC_FEATURE - peptide
LAEIDSIELSYGIK

SEQ ID NO: 171 - protein, 13 aa, synthetic construct
  REGION 1..13: note = synthetic peptide
  REGION 1..13: note = MISC_FEATURE - peptide
AEIDSIELSYGIK

SEQ ID NO: 172 - protein, 12 aa, synthetic construct
  REGION 1..12: note = synthetic peptide
  REGION 1..12: note = MISC_FEATURE - peptide
EIDSIELSYGIK

SEQ ID NO: 173 - protein, 11 aa, synthetic construct
  REGION 1..11: note = synthetic peptide
  REGION 1..11: note = MISC_FEATURE - peptide
IDSIELSYGIK

SEQ ID NO: 174 - protein, 10 aa, synthetic construct
  REGION 1..10: note = synthetic peptide
  REGION 1..10: note = MISC_FEATURE - peptide
DSIELSYGIK

SEQ ID NO: 175 - protein, 9 aa, synthetic construct
  REGION 1..9: note = synthetic peptide
  REGION 1..9: note = MISC_FEATURE - peptide
SIELSYGIK

SEQ ID NO: 176 - protein, 8 aa, synthetic construct
  REGION 1..8: note = synthetic peptide
  REGION 1..8: note = MISC_FEATURE - peptide
IELSYGIK

SEQ ID NO: 177 - protein, 7 aa, synthetic construct
  REGION 1..7: note = synthetic peptide
  SITE 1: note = MISC_FEATURE - X= E or G
  SITE 3: note = MISC_FEATURE - X= S or T
XLXYGIK

SEQ ID NO: 178 - protein, 9 aa, synthetic construct
  REGION 1..9: note = synthetic peptide
  SITE 1: note = MISC_FEATURE - Z= D or G
  SITE 2: note = MISC_FEATURE - Z= I or G
  SITE 4: note = MISC_FEATURE - Z= V or L
  SITE 5: note = MISC_FEATURE - Z= V or A
ZZSZZYGLR

SEQ ID NO: 179 - protein, 15 aa, synthetic construct
  REGION 1..15: note = synthetic peptide
  REGION 1..15: note = MISC_FEATURE - peptide
KPLAEIDSIELSYGI

SEQ ID NO: 180 - protein, 14 aa, synthetic construct
  REGION 1..14: note = synthetic peptide
  REGION 1..14: note = MISC_FEATURE - peptide
KPLAEIDSIELSYG

SEQ ID NO: 181 - protein, 13 aa, synthetic construct
  REGION 1..13: note = synthetic peptide
  REGION 1..13: note = MISC_FEATURE - peptide
KPLAEIDSIELSY

SEQ ID NO: 182 - protein, 12 aa, synthetic construct
  REGION 1..12: note = synthetic peptide
  REGION 1..12: note = MISC_FEATURE - peptide
KPLAEIDSIELS

SEQ ID NO: 183 - protein, 11 aa, synthetic construct
  REGION 1..11: note = synthetic peptide
  REGION 1..11: note = MISC_FEATURE - peptide
KPLAEIDSIEL

SEQ ID NO: 184 - protein, 10 aa, synthetic construct
  REGION 1..10: note = synthetic peptide
  REGION 1..10: note = MISC_FEATURE - peptide
KPLAEIDSIE

(19) United States
(12) Patent Application Publication
(10) Pub. No.: US 2024/0327459 A1
(43) Pub. Date: Oct. 3, 2024

(54) PEPTIDE FRAGMENTS FOR TREATMENT OF DIABETES

(71) Applicant: Follicum AB, Lund (SE)

(72) Inventors: Jan Alenfall, Lomma (SE); Maria Ekblad, Södra Sandby (SE); Pontus Dunér, Dalby (SE)

(73) Assignee: Follicum AB, Lund (SE)

(21) Appl. No.: 18/744,868

(22) Filed: Jun. 17, 2024

Related U.S. Application Data
(63) Continuation of application No. 17/289,408, filed on Apr. 28, 2021, now abandoned.

(30) Foreign Application Priority Data
Nov. 7, 2018 (EP) 18204862.9

(51) Int. Cl. C07K 7/06; A61K 38/00; A61P 3/08; C07K 7/08

(52) U.S. Cl. CPC C07K 7/06; A61P 3/08; C07K 7/08; A61K 38/00; C07K 2319/21; C07K 2319/23; C07K 2319/705

(57) ABSTRACT

The present disclosure concerns agents and their use in the treatment of endocrine, nutritional and/or metabolic diseases in a mammal. The disclosure furthermore concerns novel peptide fragments.

### CROSS REFERENCE TO RELATED APPLICATIONS

This application is a continuation of U.S. Patent Application Ser. No. 17/289,408 filed Apr. 28, 2021, which is a U.S. National Stage of international application PCT/EP2019/080563 filed Nov. 7, 2019, which claims priority to European Application No: 18204862.9 filed Nov. 7, 2018, the entire contents of each of which are incorporated herein by reference.

### SEQUENCE LISTING

The instant application contains a Sequence Listing which has been submitted in .xml format via Patent Center and is hereby incorporated by reference in its entirety. Said. xml copy, created on Jun. 13, 2024, is named 2024 Jun. 13 Sequence Listing 16FOLL-HO78303NA.xml and is 315,564 bytes in size.

### TECHNICAL FIELD

The present disclosure relates to peptides useful for treatment of diabetes and associated disorders.

### BACKGROUND

The peptide hormone insulin, which is produced by β-cells in the islets of Langerhans in the pancreas, is released in response to increasing blood glucose levels. Thus, glucose is removed from the blood by insulin dependent stimulation of glucose transporters located in the cell membranes of the target tissue, e.g. adipose tissue, skeletal muscle and liver. Insulin exerts its biological effects by binding to and activating the membrane-bound insulin receptor (IR), thereby initiating a cascade of intracellular signalling events, which regulate multiple biological processes such as glucose and lipid metabolism.

Currently, the treatment of diabetes, both type 1 and type 2 diabetes, relies primarily on insulin treatment. A complement to insulin treatment is long-acting glucagon-like peptide-1 (GLP-1) receptor agonists, i.e. derivatives that act on the same receptor as GLP-1. GLP-1 is a metabolic hormone that stimulates insulin secretion. Besides increasing insulin secretion from the pancreas in a glucose-dependent manner, GLP-1 is known to increase insulin-sensitivity in both α- and β-cells; to increase β-cell mass and insulin expression, post-translational modification, and secretion; and to decrease glucagon secretion from the pancreas. Other medications used complementary to insulin treatment for the purpose of lowering plasma glucose levels include DPP-IV inhibitors, Metformin, SGLT-2 inhibitors and sulfonylurea.

Certain drawbacks are associated with long term use of insulin, such as weight gain and increased risks of cancer and hypoglycaemia. Thus, there is a growing demand in the field for novel non-insulin compounds capable of, not only treating diabetes, by addressing insulin resistance and hyperglycemia, but also reducing associated and consequential complications.

Identification of novel compounds that can restore glucose metabolism and treat diabetes and related disorders is thus highly relevant. Multiple approaches can be contemplated, albeit none of which are obvious to the person of skill in the art.

### SUMMARY

The present inventors have found peptides which stimulate β-cell proliferation, have the ability to rescue β-cell from apoptosis induced by glucotoxic conditions, and stimulate insulin secretion from rat INS-1 β-cells as well as isolated mouse pancreatic islets. Furthermore, the present inventors found that in a glucose tolerance test, the peptides lowered plasma glucose levels in vivo and delayed onset of diabetes disease in BB lyp/lyp rats, a model for type 1 diabetes. Hence, the peptides of the present disclosure are suitable for use in the treatment of endocrine, nutritional and metabolic diseases and disorders.

In one aspect, the present disclosure relates to an agent comprising or consisting of a peptide or peptide analogue, wherein the peptide or peptide analogue comprises an amino acid sequence of the general formula:

(SEQ ID NO: 177)
X₁LX₂YGIK

wherein:
   X₁ is E or G;
   X₂ is S or T;
with the proviso that if X₂ is T then the peptide or peptide analogue comprises no more than 25 amino acids; and
with the proviso that if X₁ is E and X₂ is S, the peptide or peptide analogue comprises no more than 85 amino acid residues.

In one aspect, the present disclosure relates to an agent comprising a peptide or peptide analogue comprising or consisting of the amino acid sequence

(SEQ ID NO: 4)
DTYDGDISVVYGLR,
(SEQ ID NO: 8)
TYDGDISVVYGLR,
(SEQ ID NO: 13)
YDGDISVVYGLR,
and
(SEQ ID NO: 19)
DGDISVVYGLR.
(SEQ ID NO: 26)
GDISVVYGLR,
(SEQ ID NO: 34)
DISVVYGLR;

In one aspect, the present disclosure relates to a composition comprising the agent described herein above.

In one aspect, the present disclosure relates to a polynucleotide encoding upon expression, a peptide or peptide analogue as described herein.

In one aspect, the present disclosure relates to a vector comprising a polynucleotide as described herein.

In one aspect, the present disclosure relates to a cell comprising a polynucleotide or a vector as described herein.

In one aspect, the present disclosure relates to an agent, a polynucleotide, a vector, a cell or a composition as described herein, for use as a medicament.

In one aspect, the present disclosure relates to an agent comprising:
a) a peptide or a peptide analogue selected from the group consisting of:
   (i) a peptide comprising or consisting of an amino acid sequence of the general formula:

(SEQ ID NO: 177)
X₁LX₂YGIK

wherein:
   X₁₀ is E or G;
   X₁₂ is S or T;
with the proviso that if X₁₂ is T, the peptide or peptide analogue comprises no more than 25 amino acid residues;
   (ii) a peptide comprising or consisting of an amino acid sequence of the general formula:

(SEQ ID NO: 178)
Z₁Z₂SZ₃Z₄YGLR

wherein:
   Z₁ is D or G;
   Z₂ is I or G;
   Z₃ is V or L;
   Z₄ is V or A; and
   (iii) a peptide comprising or consisting of an amino acid sequence selected from the group consisting of VDTYDGDISVVYGL (SEQ ID NO: 3) VDTYDGDISVVYG (SEQ ID NO: 6), VDTYDGDISVVY (SEQ ID NO: 10), VDTYDGDISVV (SEQ ID NO: 15), VDTYDGDISV (SEQ ID NO: 21) and VDTYDGDIS (SEQ ID NO: 28);
b) a polynucleotide encoding upon expression, the peptide of a);
c) a vector comprising the polynucleotide of b); or
d) a cell comprising the polynucleotide of b), or the vector of c).
for use in the treatment of an endocrine disease, a nutritional disease and/or a metabolic disease in a mammal.

In one aspect, the present disclosure concerns a method for treating an endocrine disease a nutritional disease and/or a metabolic disease, the method comprising administering a therapeutically effective amount of an agent, a composition, a polynucleotide, a vector or a cell as described herein, to an individual in need thereof.

In one aspect, the present disclosure concerns the use of an agent, a composition, a polynucleotide, a vector or a cell as described herein for the manufacture of a medicament for the treatment of an endocrine disease a nutritional disease and/or a metabolic disease.

In one aspect, the present disclosure concerns a method for delaying onset of diabetes, the method comprising administering a therapeutically effective amount of an agent, a composition, a polynucleotide, a vector or a cell as described herein, to an individual in need thereof.

In one aspect, the present disclosure concerns a method for decreasing blood glucose levels, the method comprising administering a therapeutically effective amount of an agent, a composition, a polynucleotide, a vector or a cell as described herein, to an individual in need thereof.

In one aspect, the present disclosure concerns a method, e.g. an in vitro method, for improving beta cell morphology, the method comprising administering a therapeutically effective amount of an agent, a composition, a polynucleotide, a vector or a cell as described herein, to an individual in need thereof.

In one aspect, the present disclosure concerns a method for improving beta cell viability, the method comprising administering a therapeutically effective amount of an agent, a composition, a polynucleotide, a vector or a cell as described herein, to an individual in need thereof.

In one aspect, the present disclosure concerns the use of agent described herein for the preparation of a diagnostic composition for the diagnosis of a disease, disorder or damage of the pancreas in an individual.

### DESCRIPTION OF DRAWINGS

FIG. 1. FOL-005 and FOL-014 induced proliferation of β-cells

Addition of increasing concentrations of FOL-005 in solution induced increasing proliferation of INS-1 cells after 48 hours (FIG. 1A). Wells coated with FOL-005 and blocked with Bovine Serum Albumin (BSA) induced more proliferation of β-cells compared to only BSA coated control (ctrl) wells (FIG. 1B). Wells pre-coated with FOL-014 and blocked with BSA induced more proliferation compared to only BSA coated wells (FIG. 1C). Data is presented as counts per minute (CPM) relative unstimulated control (ctrl) cells. Mean±SD are presented for 10-12 different observations in each group.

FIG. 2. FOL-005 protected β-cells against glucotoxicity

INS-1 cells incubated during 48 h in 20 mM glucose displayed more apoptotic cells (Annexin V positive) compared to cells incubated at 5 mM glucose. Addition of FOL-005 to cells incubated with 20 mM glucose reduced the level of apoptotic cells compared to 20 mM glucose alone (FIG. 2A). Apoptosis measured by caspase-3 activity was increased in INS-1 cells at 20 mM compared to 5 mM glucose. Addition of FOL-005 diminished the rate of glucotoxicity-induced caspse-3 activity (FIG. 2B). Mean±SD are presented for 4-8 different observations in each group.

FIG. 3. Insulin secretion was increased from islets and β-cells following FOL-005 stimulation

FOL-005 stimulated β-cell and islet insulin secretion. Insulin release from INS-1 cells was increased after FOL-005 (6 μM) stimulation in-non glucose containing media compared to non-stimulated control (ctrl) and to a scrambled control peptide (FOL-015) (FIG. 3A). FOL-005 stimulated insulin release from INS-1 at both low (5 mM) and high (20 mM) glucose (FIG. 3B). Isolated mouse pancreatic islets stimulated with FOL-005 (6 μM) or GLP-1 (100 nM) secreted more insulin compared to unstimulated control islets (FIG. 3C). Mean±SD are presented for 5-6 different observations in each group.

FIG. 4. Insulin secretion was increased from islets and β-cells following FOL-014 stimulation

FOL-014 stimulated insulin secretion from β-cells and pancreatic islets. INS-1 cells stimulated with FOL-014 (6 μM) secreted more insulin compared to unstimulated control cells (FIG. 4A). Isolated mouse pancreatic islets stimulated with FOL-014 (6 μM) secreted more insulin compared to control islets (FIG. 4B). Addition of GLP-1 (100 nM) or FOL-014 (0.6 μM) had no effect on insulin secretion. Mean±SD are presented for 5-6 different observations in each group.

FIG. 5. The effect of FOL-014 on insulin secretion was dose dependent. Stimulation of INS-1 cells by increasing doses of FOL-014 resulted in a significant increase in insulin secretion for all concentrations tested. The insulin secretion increased in a linear fashion in the presence of FOL-014 ranging from 0.6 nM to 60 nM. Higher concentrations appeared to result in a less pronounced effect on insulin secretion. Furthermore, FOL-014 induced insulin secretion was comparable to the effect of 100 nM GLP-1. Bars represent mean values and standard error of the mean (SEM).

FIG. 6. The effect on insulin secretion of FOL-014 was glucose concentration dependent. The insulin secretion from untreated or FOL-014 exposed INS-1 cells was measured in the presence of increasing glucose concentrations. At glucose levels 5.5 mM or higher, the insulin secretion was significantly higher in the FOL-014 treated cells, as compared to untreated control cells. Bars represent mean values and standard error of the mean (SEM).

FIG. 7. FOL-005 and FOL-014 dosed together with native GLP-1 elicited an additive effect on insulin secretion. The insulin release from INS-1 cells was measured following combination treatment of GLP-1 together with FOL-005 and FOL-014 (all three peptides in a concentration of 100 nM), respectively and compared with the effect of each peptide alone. The combination of GLP-1 and FOL-014 significantly increased the insulin secretion as compared with each peptide alone. An increase was also observed for the combination of FOL-005 and GLP-1. Bars represent mean values and standard error of the mean (SEM).

FIG. 8. FOL-014 affected insulin and glucagon secretion in pancreatic islets. Two different concentrations of FOL-014 were tested and compared with the effect of 100 nM GLP-1 on isolated mouse islets in low (2.8 mM) (A, C) and high (16.7 mM) (B, D) concentrations of glucose. In the low glucose samples, the presence of FOL-014 did not increase insulin secretion, but reduced glucagon secretion as compared with control and GLP-1. In the high glucose samples, 600 nM FOL-014 and GLP-1, but not 6 μM FOL-014, significantly increased insulin secretion (B), and GLP-1 as well as both concentrations of FOL-014 efficiently reduced glucagon secretion (D). Bars represent mean values and standard error of the mean (SEM).

FIG. 9. FOL-014 lowered plasma glucose levels in vivo following a glucose injection. An intraperitoneal glucose tolerance test (IPGTT) was performed on wild type C57bl/6 mice. FOL-014 dosed at 200 nmol/kg significantly lowered the plasma glucose levels as compared to the control at 15 minutes, 30 minutes and 45 minutes (P=0.0027). At the 30 nmol/kg dose, FOL-014 lowered the glucose levels with a significant effect at 45 minutes after the glucose injection. The dotted line corresponds to mean non-fasting glucose levels. Data represents mean values and standard error of the mean (SEM). Statistical analysis was performed using student's t-test.

FIG. 10. FOL-014 delayed the onset of type-1 diabetes in BB lyp/lyp rats. BB lyp/lyp rats treated with FOL-014 showed a significant delay in the onset of diabetes defined as plasma glucose <11.1 mmol/l. Age of onset of diabetes for each rat was depicted in (A) with a significant difference between untreated and treated groups. The percentage of animals developing type 1 diabetes each day was depicted in (B) with a significant difference between groups. Error bars in (A) represent standard error of the mean (SEM).

FIG. 11. The effect on insulin secretion of peptide analogues derived from FOL-005 or FOL-014. Novel peptide analogues were tested in two separate INS-1 cell lines (A and B) for their ability to induce insulin secretion under high glucose (16.7 mM) conditions. The effect was compared with that of native GLP-1, FOL-005 and FOL-014 as well as the effect of high glucose alone. Analogues inducing insulin release below the average of the high glucose control were considered non-functional (not shown). The level of insulin secretion is depicted in black, filled bars for the novel analogues, and in contrasting patterns for the comparators. Bars represent mean values and standard error of the mean (SEM).

FIG. 12. FOL-005 and FOL-014 displayed specific distribution patterns following injection in mouse. Following subcutaneous administration of ³H-FOL-005, the highest overall levels of radioactivity were present in pancreas and at the injection site, 1 hour (A) and 2 hours (B) after injection. Accumulation of the ³H-FOL-005 is also visible in liver, kidney, salivary glands. Using Pearl Trilogy Small Animal Imaging System in vivo bio-distribution and tissue localization of Cy7.5 labelled FOL-005 (C) and FOL-014 (D) in NMRI nude mice via subcutaneous injection was investigated. Following initial control imaging, a dose of 10 nmol per mouse was administered and live imaging was performed at 5 min, 20 min, 50 min, 60 min, 2 hrs, 4 hrs, 6 hrs, 24 hrs and 48 hrs. High accumulation of both peptides was evident in the pancreatic region as well as at the injection site.

FIG. 13. FOL-056 Induce Insulin Secretion from INS-1E cells.

The peptide FOL-056 supplemented to INS-1E cells in a high (16.7 mM) glucose experimental buffer significantly increased the insulin secretion as compared with cells treated with an un-supplemented high glucose buffer. Presence of the comparator peptide, FOL-014, also resulted in a significant increase in insulin secretion. The peptides were added to the experimental buffer at a concentration of 100 nM.

FIG. 14. FOL-056 preserves the insulin secreting capacity of INS-1E cells during long-term glucotoxic conditions.

INS-1 β-cells were subjected toxic levels of glucose (20 mM) during 72 hours in the presence or absence of FOL-014 or FOL-056. For reference, cells subjected to low (5 mM) glucose were included. (A) Long term exposure to toxic levels of glucose significantly reduces the capacity of the β-cells to secrete insulin. (B) The presence of FOL-014 in the high glucose media significantly improved the insulin secreting ability of the β-cells as compared with high glucose media alone. The presence of FOL-056 in the high glucose media abolished the glucotoxic effects and retained insulin release at the same level as from β-cells in the low (5 mM) glucose treatment group.

FIG. 15. FOL-056 dosed together with native GLP-1 elicited an additive effect on insulin secretion.

The insulin release from INS-1 cells was measured following combination treatment of GLP-1 together with FOL-056 (both peptides in a concentration of 100 nM) and compared with the effect of each peptide alone. The combination of GLP-1 and FOL-056 significantly increased the insulin secretion as compared with each peptide alone. (P=0.0037 compared with GLP-1 and P=0.0003 compared with FOL-056). Data represents mean values; error bars are presented as SEM.

FIG. 16. Novel Peptide Analogues Induce Insulin Secretion from INS-1E cells. The peptides FOL-057, FOL-058 and FOL-059 supplemented to INS-1E cells in a high (16.7 mM) glucose experimental buffer increased the insulin secretion as compared with cells treated with an un-supplemented high glucose buffer. Liraglutide was included for comparison. The peptides were added to the experimental buffer at a concentration of 100 nM. Data represents mean values; error bars are presented as SEM.

FIG. 17. Novel Peptide Analogues Preserve the Insulin Secreting Capacity of INS-1E Cells During Long-term Glucotoxic Conditions.

INS-1E β-cells were subjected to toxic levels of glucose (20 mM) during 72 hours in the presence or absence of several novel peptide analogues. For reference, cells subjected to low (5 mM) glucose were included (not shown). The presence of peptide analogues in the high glucose media improved the insulin secreting ability of the β-cells as compared with high glucose media alone. Analogues inducing insulin release below the average of the high glucose control were considered non-functional (not shown). Data represents mean values; error bars are presented as SEM.

FIG. 18. FOL-056 and FOL-014 Induced Insulin Secretion from 1.2B4 Human β-cells.

The peptides FOL-056 and FOL-014 supplemented to 1.2B4 cells in a high (16.7 mM) glucose experimental buffer significantly increased the insulin secretion as compared with cells treated with an un-supplemented high glucose buffer. Liraglutide was included for comparison. The peptides were added to the experimental buffer at a concentration of 100 nM. Data represents mean values; error bars are presented as SEM.

FIG. 19. FOL-056 Induced Insulin Secretion from Human Islets.

The peptide FOL-056 supplemented to freshly isolated human islets from two separate donors in a high (16.7 mM) glucose experimental buffer significantly increased the insulin secretion as compared with cells treated with an un-supplemented high glucose buffer. The effect of Liraglutide was tested for comparison. FOL-056 and liraglutide was added to the experimental buffer at a concentration of 1 and 100 nM respectively. Data represents mean values; error bars are presented as SEM.

FIG. 20. FOL-056 Retained the Capacity of Insulin Secretion in Response to Elevated Glucose Levels in a Diet Induced Obese Mouse Model.

Following 12 weeks of dosing in c57BI6 mice on high fat diet, the acute insulin response (AIR), measured as the increase in plasma insulin after a glucose injection, was significantly higher in mice treated with FOL-056 as compared with the untreated control group (P=0.01). Data represents mean values; error bars are presented as SEM.

FIG. 21. Dosing with FOL-014 or FOL-056 Reduced HbA1c in a Diabetic Mouse Model.

Analysis of whole blood samples collected from db/db mice following 4 weeks of dosing showed a significantly lower HbA1c in animals treated with FOL-014 (P=0.0015) or FOL-056 (P=0.0028) as compared with untreated control animals. Data represents mean values; error bars are presented as SEM.

### DETAILED DESCRIPTION

The disclosure is as defined in the claims.

In one aspect, the present disclosure concerns an agent comprising or consisting of:
a) a peptide or peptide analogue, wherein the peptide or peptide analogue comprises an amino acid sequence of the general formula:

(SEQ ID NO: 177)
X₁LX₂YGIK

wherein:
   X₁ is E or G;
   X₂ is S or T;
with the proviso that if X₂ is T, the peptide or peptide analogue comprises no more than 25 amino acids; and
with the proviso that if X₁ is E and X₂ is S, the peptide or peptide analogue comprises no more than 85 amino acid residues;
b) a polynucleotide encoding upon expression, the peptide of a);
c) a vector comprising the polynucleotide of b); or
d) a cell comprising the polynucleotide of b), or the vector of c).

In one embodiment, the present disclosure concerns a peptide or a peptide analogue comprising an amino acid sequence of the general formula:

(SEQ ID NO: 177)
X1LX2YGIK

wherein:
X₁ is E or G;
X₂ is S or T;
with the proviso that if X2 is T, the peptide or peptide analogue comprises no more than 25 amino acids; and
with the proviso that if X1 is E and X2 is S, the peptide or peptide analogue comprises no more than 85 amino acid residues.

In one embodiment, the present disclosure concerns a polynucleotide encoding upon expression, a peptide or peptide analogue as described herein.

In one embodiment, the present disclosure concerns a vector comprising a polynucleotide as described herein.

In one embodiment, the present disclosure concerns a cell comprising a polynucleotide as described herein. In one embodiment, the present disclosure concerns a cell comprising a vector as described herein.

In one embodiment, the present disclosure concerns an agent comprising:
a) a peptide, wherein the peptide is selected from the group consisting of:
   i) a peptide comprising or consisting of the amino acid sequence of SEQ ID NO: 170,171,172,173,174,175, 176, 177, 178, 179, 180, 181,182,183 and 184;
   ii) a biologically active sequence variant of any one of the peptides of i), wherein any one amino acid has been altered for another proteinogenic or non-proteinogenic amino acid, with the proviso that no more than five amino acids are so altered;
   iii) a biologically active fragment of the peptide of any one of i) or ii), wherein the fragment comprises at least 10 consecutive amino acids of any one of i) or ii);
b) a polynucleotide encoding upon expression, the peptide of a);
c) a vector comprising the polynucleotide of b); or
d) a cell comprising the polynucleotide of b), or the vector of c).

In one embodiment, the present disclosure concerns an agent comprising a peptide, wherein the peptide is selected from the group consisting of a peptide comprising or consisting of the amino acid sequence of SEQ ID NO: 170,171,172,173,174,175, 176, 177, 178, 179, 180, 181,182,183 and 184.

In one embodiment, the present disclosure concerns a biologically active sequence variant of any one of the peptides described herein, wherein any one amino acid has been altered for another proteinogenic or non-proteinogenic amino acid, with the proviso that no more than five amino acids are so altered.

In one embodiment, the present disclosure concerns an agent comprising:
a) a peptide, wherein the peptide comprises or consists of an amino acid sequence selected from the group consisting of DTYDGDISVVYGLR (SEQ ID NO: 4), TYDGDISVVYGLR (SEQ ID NO: 8), YDGDISVVYGLR (SEQ ID NO: 13), and DGDISVVYGLR (SEQ ID NO: 19). GDISVVYGLR (SEQ ID NO: 26), DISVVYGLR (SEQ ID NO: 34);
b) a polynucleotide encoding upon expression, the peptide of a);
c) a vector comprising the polynucleotide of b); and
d) a cell comprising the polynucleotide of b), or the vector of c).

In one embodiment, the present disclosure concerns an agent comprising a peptide, wherein the peptide comprises or consists of an amino acid sequence selected from the group consisting of DTYDGDISVVYGLR (SEQ ID NO: 4), TYDGDISVVYGLR (SEQ ID NO: 8), YDGDISVVYGLR (SEQ ID NO: 13), DGDISVVYGLR (SEQ ID NO: 19), GDISVVYGLR (SEQ ID NO: 26) and DISVVYGLR (SEQ ID NO: 34).

In one embodiment, the present disclosure concerns a peptide comprising an amino acid sequence of the general formula:

(SEQ ID NO: 178)
Z₁Z₂SZ₃Z₄YGLR

wherein:
   Z₁ is D or G;
   Z₂ is I or G;
   Z₃ is V or L;
   Z₄ is V or A.

The term ‘absent’ as used herein, e.g. “X₆ is C, I or absent” is to be understood as that the amino acid residues directly adjacent to the absent amino acid are directly linked to each other by a conventional amide bond.

The term “peptide analogue” described herein refers to an amino acid sequence non-naturally occurring, or a naturally occurring amino acid sequence that has been modified.

The term ‘amino acid’ as used herein includes the standard twenty genetically-encoded amino acids and their corresponding stereoisomers in the ‘D’ form (as compared to the natural ‘L’ form), omega-amino acids and other naturally-occurring amino acids, unconventional amino acids (e.g., α,α-disubstituted amino acids, N-alkyl amino acids, etc.) and chemically derivatized amino acids (see below).

When an amino acid is being specifically enumerated, such as ‘alanine’ or ‘Ala’ or ‘A’, the term refers to both L-alanine and D-alanine unless explicitly stated otherwise. Other unconventional amino acids may also be suitable components for peptides of the present disclosure, as long as the desired functional property is retained by the peptide. For the peptides shown, each encoded amino acid residue, where appropriate, is represented by a single letter designation, corresponding to the trivial name of the conventional amino acid.

Chemical derivatives of one or more amino acids may be achieved by reaction with a functional side group. Such derivatives include, for example, those molecules in which free amino groups have been derivatized to form amine hydrochlorides, p-toluene sulphonyl groups, carboxybenzoxy groups, t-butyloxycarbonyl groups, chloroacetyl groups or formyl groups. Free carboxyl groups may be derivatized to form salts, methyl and ethyl esters or other types of esters and hydrazides. Free hydroxyl groups may be derivatized to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

Some of the peptides of the disclosure shares amino acid sequence similarity with a sub-region of naturally occurring osteopontin proteins. In some embodiments, said peptide may be regarded as an active fragment of a naturally-occurring osteopontin protein or a variant of such as a fragment.

Some of the peptides of the disclosure shares amino acid sequence similarity with a sub-region of naturally occurring tenascin proteins. In some embodiments, said peptide may be regarded as an active fragment of a naturally-occurring tenascin protein or a variant of such as a fragment.

By “fragment”, at least 5 contiguous amino acids of the amino acid sequence are included, for example at least 6, 7, 8, 9, 10, 11, 12, 13, 14 or 15 contiguous amino acids of the amino acid sequence. Thus, the fragment may be 15 or fewer amino acids in length, for example 14, 13, 12, 11, 10, 9, 8, 7, 6 or 5 amino acids in length

In one embodiment, said peptide is of no more than no more than 85, such as no more than 80, such as no more than 75, such as no more than 70, such as no more than 65, such as no more than 60, such as nor more than 55, such as no more than 50, such as no more than 55, such as no more than 40 amino acids, such as no more than 35, such as no more than 30, such as no more than 28, such as no more than 26, such as no more than 24, such as no more than 22, such as no more than 20, such as no more than 19, such as no more than 18, such as no more than 17, such as no more than 16, such as no more than 15, such as no more than 14, such as no more than 13, such as no more than 12, such as no more than 11, such as no more than 10 amino acids in length.

In another embodiment, said peptide is between 5 and 30 amino acids in length, such as between 5 and 20, such as between 8 and 20, such as between 8 and 16, such as between 10 and 15 amino acids in length.

In yet another embodiment, said fragment comprises 15 or fewer amino acids in length, such as fewer than 14 amino acids, such as fewer than 13 amino acids, such as fewer than 12 amino acids, such as fewer than 11 amino acids, such as fewer than 10 amino acids, such as fewer than 9 amino acids, such as fewer than 8 amino acids, such as fewer than 7 amino acids, such as fewer than 6 amino acids, such as fewer than 5 amino acids in length.

The term “variant” refers to a peptide that does not share 100% amino acid sequence identity with the parent peptide, i.e. one or more amino acids must be mutated. “Mutated” refers to altering an amino acid at a specified position in the parent peptide. For example, an amino acid at a specified position may be deleted, altered, substituted or may be the site of an insertion/addition of one or more amino acids. It will be appreciated by persons skilled in the art that the substitutions may be conservative or non-conservative.

In one embodiment, said peptide variant comprises or consists of a sequence wherein no more than five amino acids are altered for another proteinogenic or non-proteinogenic amino acid, such as no more than 4 amino acids, such as no more than 3 amino acids, such as no more than 2 amino acids, such as no more than 1 amino acid is altered. In one embodiment, one or more amino acids are conservatively substituted. “Conservatively substituted” refers to a substitution of one amino acid with another with similar properties (size, hydrophobicity, etc.), such that the function of the peptide is not significantly altered. Thus, by “conservative substitutions” is intended combinations such as Gly, Ala; Val, Ile, Leu; Asp, Glu; Asn, Gln; Ser, Thr; Lys, Arg; and Phe, Tyr.

In another embodiment, said peptide comprises or consists of one or more additional amino acids, inserted at the N- and/or C-terminus and/or internally within the sequence. In one embodiment, at least 2 additional amino acids, such as at least 3, such as at least 4, such as at least 5, such as at least 6, such as at least 7, such as at least 8, such as at least 9, such as at least 10, such as at least 15 or such as at least 20 additional amino acids are inserted. The additional amino acids may be the amino acids from the corresponding positions of the wildtype human osteopontin (SEQ ID NO: 66) or from the corresponding positions of the wildtype murine osteopontin (SEQ ID NO: 134). The term “corresponding positions” of the wildtype osteopontin we mean that the additional amino acids are the same as those present in the equivalent position in the above wildtype osteopontin (if one imagines that the amino acid sequence of SEQ ID NO:1 replaces the sequence underlined in italics in SEQ ID NO:66

In another embodiment, the peptide is selected from the group consisting of SEQ ID NO: 1, 136, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 67, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 135, 137, 138, 139, 141, 142, 143, 144, 145, 146, 147, 148, 149, 150, 151, 152, 153, 154, 155, 156, 157, 158, 159, 160, 161, 167, 168, 169, 171,171,172,173,174,175, 176, 177, 178, 179, 180, 181, 182, 183 and 184;

i. 15-amino acid peptides:
SEQ ID NO: 1
VDTYDGDISVVYGLR
SEQ ID NO: 2
VDTYDGDISVVYGLS
ii. 14-amino acid peptides:
SEQ ID NO: 3
VDTYDGDISVVYGL
SEQ ID NO: 4
DTYDGDISVVYGLR
SEQ ID NO: 5
TYDGDISVVYGLRS
iii. 13-amino acid peptides:
SEQ ID NO: 6
VDTYDGDISVVYG
SEQ ID NO: 7
DTYDGDISVVYGL
SEQ ID NO: 8
TYDGDISVVYGLR
SEQ ID NO: 9
YDGDISVVYGLRS
iv. 12-amino acid peptides:
SEQ ID NO: 10
VDTYDGDISVVY
SEQ ID NO: 11
DTYDGDISVVYG
SEQ ID NO: 12
TYDGDISVVYGL
SEQ ID NO: 13
YDGDISVVYGLR
SEQ ID NO: 14
DGDISVVYGLRS
v. 11-amino acid peptides:
SEQ ID NO: 15
VDTYDGDISVV
SEQ ID NO: 16
DTYDGDISVVY
SEQ ID NO: 17
TYDGDISVVYG
SEQ ID NO: 18
YDGDISVVYGL
SEQ ID NO: 19
DGDISVVYGLR
SEQ ID NO: 20
GDISVVYGLRS
vi. 10-amino acid peptides:
SEQ ID NO: 21
VDTYDGDISV
SEQ ID NO: 22
DTYDGDISVV
SEQ ID NO: 23
TYDGDISVVY
SEQ ID NO: 24
YDGDISVVYG
SEQ ID NO: 25
DGDISVVYGL
SEQ ID NO: 26
GDISVVYGLR
SEQ ID NO: 27
DISVVYGLRS
vii. 9-amino acid peptides:
SEQ ID NO: 28
VDTYDGDIS
SEQ ID NO: 29
DTYDGDISV
SEQ ID NO: 30
TYDGDISVV
SEQ ID NO: 31
YDGDISVVY
SEQ ID NO: 32
DGDISVVYG
SEQ ID NO: 33
GDISVVYGL
SEQ ID NO: 34
DISVVYGLR
SEQ ID NO: 35
ISVVYGLRS
viii. 8-amino acid peptides:
SEQ ID NO: 36
VDTYDGDI
SEQ ID NO: 37
DTYDGDIS
SEQ ID NO: 38
TYDGDISV
SEQ ID NO: 39
YDGDISVV
SEQ ID NO: 40
DGDISVVY
SEQ ID NO: 41
GDISVVYG
SEQ ID NO: 42
DISVVYGL
SEQ ID NO: 43
ISVVYGLR
ix. 7-amino acid peptides:
SEQ ID NO: 44
VDTYDGD
SEQ ID NO: 45
DTYDGDI
SEQ ID NO: 46
TYDGDIS
SEQ ID NO: 47
YDGDISV
SEQ ID NO: 48
DGDISVV
SEQ ID NO: 49
GDISVVY
SEQ ID NO: 50
DISVVYG
SEQ ID NO: 51
ISVVYGL
x. 6-amino acid peptides:
SEQ ID NO: 52
DTYDGD
SEQ ID NO: 53
TYDGDI
SEQ ID NO: 54
YDGDIS
SEQ ID NO: 55
DGDISV
SEQ ID NO: 56
GDISVV
SEQ ID NO: 57
DISVVY
SEQ ID NO: 58
ISVVYG
xi. 5-amino acid peptides:
SEQ ID NO: 59
TYDGD
SEQ ID NO: 60
YDGDI
SEQ ID NO: 61
DGDIS
SEQ ID NO: 62
GDISV
SEQ ID NO: 63
DISVV
SEQ ID NO: 64
ISVVY
SEQ ID NO: 65
SVVYG
xii. 16-amino acid peptide:
SEQ ID NO: 67
VDTYDGRGDSVVYGLR
xiii. 15-amino acid peptides:
SEQ ID NO: 69
VDVPNGDISLAYGLR
SEQ ID NO: 70
DVPNGDISLAYGLRS
xiv. 14-amino acid peptides:
SEQ ID NO: 71
VDVPNGDISLAYGL
SEQ ID NO: 72
DVPNGDISLAYGLR
SEQ ID NO: 73
VPNGDISLAYGLRS
xv. 13-amino acid peptides:
SEQ ID NO: 74
VDVPNGDISLAYG
SEQ ID NO: 75
DVPNGDISLAYGL
SEQ ID NO: 76
VPNGDISLAYGLR
SEQ ID NO: 77
PNGDISLAYGLRS
xvi. 12-amino acid peptides:
SEQ ID NO: 78
VDVPNGDISLAY
SEQ ID NO: 79
DVPNGDISLAYG
SEQ ID NO: 80
VPNGDISLAYGL
SEQ ID NO: 81
PNGDISLAYGLR
SEQ ID NO: 82
NGDISLAYGLRS
xvii. 11-amino acid peptides:
SEQ ID NO: 83
VDVPNGDISLA
SEQ ID NO: 84
DVPNGDISLAY
SEQ ID NO: 85
VPNGDISLAYG
SEQ ID NO: 86
PNGDISLAYGL
SEQ ID NO: 87
NGDISLAYGLR
SEQ ID NO: 88
GDISLAYGLRS
xviii. 10-amino acid peptides:
SEQ ID NO: 89
VDVPNGDISL
SEQ ID NO: 90
DVPNGDISLA
SEQ ID NO: 91
VPNGDISLAY
SEQ ID NO: 92
PNGDISLAYG
SEQ ID NO: 93
NGDISLAYGL
SEQ ID NO: 94
GDISLAYGLR
SEQ ID NO: 95
DISLAYGLRS
xix. 9-amino acid peptides:
SEQ ID NO: 96
VDVPNGDIS
SEQ ID NO: 97
DVPNGDISL
SEQ ID NO: 98
VPNGDISLA
SEQ ID NO: 99
PNGDISLAY
SEQ ID NO: 100
NGDISLAYG
SEQ ID NO: 101
GDISLAYGL
SEQ ID NO: 102
DISLAYGLR
SEQ ID NO: 103
ISLAYGLRS
xx. 8-amino acid peptides:
SEQ ID NO: 104
VDVPNGDI
SEQ ID NO: 105
DVPNGDIS
SEQ ID NO: 106
VPNGDISL
SEQ ID NO: 107
PNGDISLA
SEQ ID NO: 108
NGDISLAY
SEQ ID NO: 109
GDISLAYG
SEQ ID NO: 110
DISLAYGL
SEQ ID NO: 111
ISLAYGLR
xxi. 7-amino acid peptides:
SEQ ID NO: 112
VDVPNGD
SEQ ID NO: 113
DVPNGDI
SEQ ID NO: 114
VPNGDIS
SEQ ID NO: 115
PNGDISL
SEQ ID NO: 116
NGDISLA
SEQ ID NO: 117
GDISLAY
SEQ ID NO: 118
DISLAYG
SEQ ID NO: 119
ISLAYGL
xxii. 6-amino acid peptides:
SEQ ID NO: 120
DVPNGD
SEQ ID NO: 121
VPNGDI
SEQ ID NO: 122
PNGDIS
SEQ ID NO: 123
NGDISL
SEQ ID NO: 124
GDISLA
SEQ ID NO: 125
DISLAY
SEQ ID NO: 126
ISLAYG
xxiii. 5-amino acid peptides:
SEQ ID NO: 127
VPNGD
SEQ ID NO: 128
PNGDI
SEQ ID NO: 129
NGDIS
SEQ ID NO: 130
GDISL
SEQ ID NO: 131
DISLA
SEQ ID NO: 132
ISLAY
SEQ ID NO: 133
SLAYG
xxiv. 16-amino acid peptides:
SEQ ID NO: 136
KPLAEIDSIELSYGIK
SEQ ID NO: 137
GDPNDGRGDSVVYGLR
xxv. 15--amino acid peptides:
SEQ ID NO: 138
VDTYDGGISVVYGLR
SEQ ID NO: 139
VDTYDGDGSVVYGLR
xxvi. 16-amino acid peptides:
SEQ ID NO: 141
KCLAECDSIELSYGIK
xxvii. 8--amino acid peptides:
SEQ ID NO: 142
CLAEIDSC
xxviii. 18-amino acid peptides:
SEQ ID NO: 143
CFKPLAEIDSIECSYGIK
xxix. 16--amino acid peptides:
SEQ ID NO: 144
KPLAEDISIELSYGIK
SEQ ID NO: 145
KPLAEISDIELSYGIK
SEQ ID NO: 146
KPLAEIGDIELSYGIK
xxx. 15-amino acid peptides:
SEQ ID NO: 147
KPLAEGDIELSYGIK
xxxi. 13--amino acid peptides:
SEQ ID NO: 148
KPLAEIELSYGIK
xxxii. 16--amino acid peptides:
SEQ ID NO: 149
KPLAEIDSIELTYGIK
SEQ ID NO: 150
KPLAEIDGIELSYGIK
SEQ ID NO: 151
KPLAEIDGIELTYGIK
SEQ ID NO: 152
KPLAEIGSIELSYGIK
SEQ ID NO: 153
KGLAEIDSIELSYGIK
SEQ ID NO: 154
KPLAGIDSIGLSYGIK
SEQ ID NO: 155
KCLAEIDSCELSYGIK
xxxiii. 13--amino acid peptides:
SEQ ID NO: 156
CFKPLAEIDSIEC
xxxiv. 15-amino acid peptides:
SEQ ID NO: 157
VDVPEGDISLAYGLR
SEQ ID NO: 158
LDGLVRAYDNISPVG
xxxv. 14-amino acid peptides:
SEQ ID NO: 159
GDPNGDISVVYGLR
xxxvi. 15-amino acid peptides:
SEQ ID NO: 160
VDVPNGDISLAYRLR
SEQ ID NO: 161
VDVPEGDISLAYRLR
SEQ ID NO: 167
V(beta-D)TYDGDISVVYGLR
SEQ ID NO: 168
VDTY (beta-D) GDISVVYGLR
SEQ ID NO: 169
VDTYDG(beta-D)ISVVYGLR
xxxvii. 14-amino acid peptides:
SEQ ID NO: 170
LAEIDSIELSYGIK
xxxviii. 13-amino acid peptides:
SEQ ID NO: 171
AEIDSIELSYGIK
xxxix. 12-amino acid peptides:
SEQ ID NO: 172
EIDSIELSYGIK
xl. 11-amino acid peptides:
SEQ ID NO: 173
IDSIELSYGIK
xli. 10-amino acid peptides:
SEQ ID NO: 174
DSIELSYGIK
xlii. 9-amino acid peptides:
SEQ ID NO: 175
SIELSYGIK
xliii. 8-amino acid peptides:
SEQ ID NO: 176
IELSYGIK
xliv. 15-amino acid peptides:
SEQ ID NO: 179
KPLAEIDSIELSYGI
xlv. 14-amino acid peptides:
SEQ ID NO: 180
KPLAEIDSIELSYG
xlvi. 13-amino acid peptides:
SEQ ID NO: 181
KPLAEIDSIELSY
xlvii. 12-amino acid peptides:
SEQ ID NO: 182
KPLAEIDSIELS
xlviii. 10-amino acid peptides:
SEQ ID NO: 183
KPLAEIDSIEL
xlix. 9-amino acid peptides:
SEQ ID NO: 184
KPLAEIDSIE

In one embodiment said peptide is derived from osteopontin, such as a mammalian osteopontin variant and/or fragment.

In one embodiment, said peptide is non-naturally occurring, such as a peptide comprising non-proteinogenic amino acid residues.

In some embodiments, said peptide is further conjugated to a moiety, which may be selected from the group consisting of PEG, monosaccharides, fluorophores, chromophores, radioactive compounds, and cell-penetrating peptides. In one embodiment, the fluorophore is selected from the group consisting of Lucifer yellow, biotin, 5,6-carboxyltetramethylrhodamine (TAMRA), indodicarbocyanine (C5) Alexa Fluor® 488, Alexa Fluor® 532, Alexa Fluor® 647, ATTO 488, ATTO 532, 6-carboxyfluorescein (6-FAM), Alexa Fluor® 350, DY-415, ATTO 425, ATTO 465, Bodipy® FL, fluorescein isothiocyanate, Oregon Green® 488, Oregon Green® 514, Rhodamine Green™, 5′-Tetrachloro-Fluorescein, ATTO 520, 6-carboxy-4′,5′-dichloro-2′,7′-dimethoxyfluoresceine, Yakima Yellow™ dyes, Bodipy® 530/550, hexachloro-fluorescein, Alexa Fluor® 555, DY-549, Bodipy® TMR-X, cyanine phosphoramidites (cyanine 3, cyanine 3.5, cyanine 5, cyanine 5.5, cyanine 7.5), ATTO 550, Rhodamine Red™, ATTO 565, Carboxy-X-Rhodamine, Texas Red (Sulforhodamine 101 acid chloride), LightCycler® Red 610, ATTO 594, DY-480-XL, DY-610, ATTO 610, LightCycler® Red 640, Bodipy 630/650, ATTO 633, Bodipy 650/665, ATTO 647N, DY-649, LightCycler® Red 670, ATTO 680, LightCycler® Red 705, DY-682, ATTO 700, ATTO 740, DY-782, IRD 700, IRD 800, CAL Fluor® Gold 540 nm, CAL Fluor® Gold 522 nm, CAL Fluor® Gold 544 nm, CAL Fluor® Orange 560 nm, CAL Fluor® Orange 538 nm, CAL Fluor® Orange 559 nm, CAL Fluor® Red 590 nm, CAL Fluor® Red 569 nm, CAL Fluor® Red 591 nm, CAL Fluor® Red 610 nm, CAL Fluor® Red 590 nm, CAL Fluor® Red 610 nm, CAL Fluor® Red 635 nm, Quasar® 570 nm, Quasar® 548 nm, Quasar® 566 nm (Cy 3), Quasar® 670 nm, Quasar® 647 nm, Quasar® 670 nm, Quasar® 705 nm, Quasar® 690 nm, Quasar® 705 nm (Cy 5.5), Pulsar® 650 Dyes, SuperRox® Dyes.).

In another embodiment, said peptide is further modified such as being glycosylated or by PEGylation, amidation, esterification, acylation, acetylation and/or alkylation.

In one embodiment, said peptide comprises or consists of tandem repeats, which may comprise or consist of the amino acid sequence of any one or more of the sequences as described herein.

In one embodiment, said peptide is cyclic. The cyclic structure may be achieved by any suitable method of synthesis. Thus, heterodetic linkages may include, but are not limited to formation via disulphide, cysteine, alkylene or sulphide bridges.

In a further embodiment, the peptide comprises or consists of a fusion. For example, the peptide may comprise a fusion of the amino acid sequence of SEQ ID NO: 1 or 136.

The term ‘fusion’ of a peptide relates to an amino acid sequence corresponding to, for example, SEQ ID NO: 1 or 136 (or a fragment or variant thereof) fused to any other peptide. For example, the said peptide may be fused to a polypeptide such as glutathione-S-transferase (GST) or protein A in order to facilitate purification of said peptide. Examples of such fusions are well known to those skilled in the art. Similarly, the said peptide may be fused to an oligo-histidine tag such as His6 or to an epitope recognised by an antibody such as the well-known Myc tag epitope. Fusions to any variant or derivative of said peptide are also included in the scope of the disclosure.

Alternatively, the fused portion may be a lipophilic molecule or peptide domain that is capable of promoting cellular uptake of the polypeptide, as known to those skilled in the art.

### Novel Peptides

In one embodiment, the present disclosure relates to a peptide comprising or consisting of an amino acid sequence selected from the group consisting of LAEIDSIELSYGIK (SEQ ID NO: 170), AEIDSIELSYGIK (SEQ ID NO: 171), EIDSIELSYGIK (SEQ ID NO: 172), IDSIELSYGIK (SEQ ID NO: 173), DSIELSYGIK (SEQ ID NO: 174), SIELSYGIK (SEQ ID NO: 175), IELSYGIK (SEQ ID NO: 148), KPLAEIDSIELTYGIK (SEQ ID NO: 176), or a variant or fragment thereof.

In one embodiment, the peptide or peptide analogue comprises or consists of an amino acid sequence selected from the group consisting of KPLAEIDSIELSYGI (SEQ ID NO: 179), KPLAEIDSIELSYG (SEQ ID NO: 180), KPLAEIDSIELSY (SEQ ID NO: 181), KPLAEIDSIELS (SEQ ID NO: 182), KPLAEIDSIEL (SEQ ID NO: 183), KPLAEIDSIE (SEQ ID NO: 184), or a variant of fragment thereof.

In one embodiment, the present disclosure relates to the agent comprising a peptide, wherein the peptide comprises or consists of the amino acid sequence LAEIDSIELSYGIK (SEQ ID NO: 170), or a variant or fragment thereof.

In one embodiment, the present disclosure relates to the agent comprising a peptide, wherein the peptide comprises or consists of the amino acid sequence AEIDSIELSYGIK (SEQ ID NO: 171), or a variant or fragment thereof.

In one embodiment, the present disclosure relates to the agent comprising a peptide, wherein the peptide comprises or consists of the amino acid sequence EIDSIELSYGIK (SEQ ID NO: 172), or a variant or fragment thereof.

In one embodiment, the present disclosure relates to the agent comprising a peptide, wherein the peptide comprises or consists of the amino acid sequence IDSIELSYGIK (SEQ ID NO: 173), or a variant or fragment thereof.

In one embodiment, the present disclosure relates to the agent comprising a peptide, wherein the peptide comprises or consists of the amino acid sequence DSIELSYGIK (SEQ ID NO: 174), or a variant or fragment thereof.

In one embodiment, the present disclosure relates to the agent comprising a peptide, wherein the peptide comprises or consists of the amino acid sequence SIELSYGIK (SEQ ID NO: 175), or a variant or fragment thereof.

In one embodiment, the present disclosure relates to the agent comprising a peptide, wherein the peptide comprises or consists of the amino acid sequence IELSYGIK (SEQ ID NO: 176), or a variant or fragment thereof.

In one embodiment, the present disclosure relates to the agent comprising a peptide, wherein the peptide comprises or consists of the amino acid sequence KPLAEIDSIELSYGI (SEQ ID NO: 179), or a variant of fragment thereof.

In one embodiment, the present disclosure relates to the agent comprising a peptide, wherein the peptide comprises or consists of the amino acid sequence, KPLAEIDSIELSYG (SEQ ID NO: 180), or a variant of fragment thereof.

In one embodiment, the present disclosure relates to the agent comprising a peptide, wherein the peptide comprises or consists of the amino acid sequence KPLAEIDSIELSY (SEQ ID NO: 181), or a variant of fragment thereof.

In one embodiment, the present disclosure relates to the agent comprising a peptide, wherein the peptide comprises or consists of the amino acid sequence KPLAEIDSIELS (SEQ ID NO: 182), or a variant of fragment thereof.

In one embodiment, the present disclosure relates to the agent comprising a peptide, wherein the peptide comprises or consists of the amino acid sequence KPLAEIDSIEL (SEQ ID NO: 183), or a variant of fragment thereof, or a variant of fragment thereof.

In one embodiment, the present disclosure relates to the agent comprising a peptide, wherein the peptide comprises or consists of the amino acid KPLAEIDSIE (SEQ ID NO: 184), or a variant of fragment thereof, or a variant of fragment thereof.

In one embodiment, the present disclosure relates to an agent comprising:
a) a peptide or peptide analogue comprising or consisting of the amino acid sequence DTYDGDISVVYGLR (SEQ ID NO: 4), TYDGDISVVYGLR (SEQ ID NO: 8), YDGDISVVYGLR (SEQ ID NO: 13), and DGDISVVYGLR (SEQ ID NO: 19). GDISVVYGLR (SEQ ID NO: 26), DISVVYGLR (SEQ ID NO: 34);
b) a polynucleotide encoding upon expression, the peptide of a);
c) a vector comprising the polynucleotide of b); or
d) a cell comprising the polynucleotide of b), or the vector of c).

In one embodiment, the present disclosure relates to an agent comprising a peptide or peptide analogue comprising or consisting of the amino acid sequence

(SEQ ID NO: 4)
DTYDGDISVVYGLR,
(SEQ ID NO: 8)
TYDGDISVVYGLR,
(SEQ ID NO: 13)
YDGDISVVYGLR,
and
(SEQ ID NO: 19)
DGDISVVYGLR.
(SEQ ID NO: 26)
GDISVVYGLR,
(SEQ ID NO: 34)
DISVVYGLR.

In some embodiments, said variant comprises or consists of a sequence wherein any one amino acid has been altered for another proteinogenic or non-proteinogenic amino acid, with the proviso that no more than five amino acids are so altered, such as no more than 4 amino acids, such as no more than 3 amino acids, such as no more than 2 amino acids, such as no more than 1 amino acid is altered. In some embodiments, one or more amino acids are conservatively substituted.

In some embodiments, said peptide comprises or consists of one or more additional amino acids, inserted at the N- and/or C-terminus and/or internally within the sequence. In one embodiment, at least 2 additional amino acids, such as at least 3, such as at least 4, such as at least 5, such as at least 6, such as at least 7, such as at least 8, such as at least 9, such as at least 10, such as at least 15 or such as at least 20 additional amino acids are inserted.

In one embodiment, the peptide or peptide analogue comprises an amino acid residue P at the N-terminus

In some embodiments, said peptide is no more than 85, such as no more than 80, such as no more than 75, such as no more than 70, such as no more than 65, such as no more than 60, such as nor more than 55, such as no more than 50, such as no more than 55, such as no more than 40 amino acids, such as no more than 35, such as no more than 30, such as no more than 28, such as no more than 26, such as no more than 24, such as no more than 22, such as no more than 20, such as no more than 19, such as no more than 18, such as no more than 17, such as no more than 16, such as no more than 15, such as no more than 14, such as no more than 13, such as no more than 12, such as no more than 11, such as no more than 10 amino acids in length.

In some embodiments, said peptide is further conjugated to a moiety, which may be selected from the group consisting of PEG, monosaccharides, fluorophores, chromophores, radioactive compounds, and cell-penetrating peptides.

In one embodiment, said peptide is further modified such as being glycosylated or by PEGylation, amidation, esterification, acylation, acetylation and/or alkylation.

In some embodiments, said peptide comprises or consists of tandem repeats, which may comprise or consist of the amino acid sequence of any one or more of the sequences as described herein above.

In one embodiment, said peptide is cyclic. The cyclic structure may be achieved by any suitable method of synthesis. Thus, heterodetic linkages may include, but are not limited to formation via, cysteine, disulphide, alkylene or sulphide bridges.

### Indications

The agents, the peptides or peptide analogues, the compositions, the polynucleotides, the vectors or the cells of the present disclosure are suitable for use in the treatment of endocrine, nutritional and metabolic diseases and disorders.

In one embodiment, the mammal in need of treatment of an endocrine disease, a nutritional disease and/or a metabolic disease is a human.

In some embodiments, the endocrine disease, nutritional disease and/or metabolic disease is selected from the group consisting of diabetes mellitus, type 1 diabetes mellitus, type 2 diabetes mellitus, malnutrition-related diabetes mellitus, disorders of glucose regulation and pancreatic internal secretion, insulin resistance syndrome, impaired glucose tolerance, hyperglycemia, hyperinsulinemia, and any combinations thereof.

In some embodiments, the endocrine disease, nutritional disease and/or metabolic disease is selected from the group consisting of diabetes mellitus, disorders of the thyroid gland, disorders of glucose regulation and pancreatic internal secretion, disorders of endocrine glands, malnutrition, nutritional deficiencies, obesity, hyperalimentation, and metabolic disorders.

In one embodiment, diabetes mellitus is selected from the group consisting of type 1 diabetes mellitus, type 2 diabetes mellitus, malnutrition-related diabetes mellitus, specified diabetes mellitus, and unspecified diabetes mellitus.

In one embodiment, disorders of glucose regulation and pancreatic internal secretion are selected from the group consisting of nondiabetic hypoglycaemic coma and disorders of pancreatic internal secretion.

In one embodiment, disorders of obesity and hyperalimentation are selected from the group consisting of localized adiposity, hyperalimentation, and sequelae of hyperalimentation.

In one embodiment, disorders of nutritional deficiencies are selected from the group consisting of disorders of aromatic amino-acid metabolism, disorders of branched-chain amino-acid metabolism and fatty-acid metabolism, disorders of amino-acid metabolism, lactose intolerance, disorders of carbohydrate metabolism, disorders of sphingolipid metabolism, disorders of lipid storage disorders, disorders of glycosaminoglycan metabolism, disorders of glycoprotein metabolism, disorders of lipoprotein metabolism, lipidaemias, disorders of purine and pyrimidine metabolism, disorders of porphyrin and bilirubin metabolism, disorders of mineral metabolism, cystic fibrosis, amyloidosis, volume depletion, disorders of fluid, electrolyte and acid-base balance, and postprocedural endocrine and metabolic disorders.

### Compositions

In one aspect, the present disclosure relates to a composition comprising the agent described herein. The composition may be a pharmaceutical composition.

In one aspect, the present disclosure relates to an agent comprising or consisting of:
a) a peptide or a peptide analogue selected from the group consisting of
   (i) a peptide comprising or consisting of an amino acid sequence of the general formula:

(SEQ ID NO: 177)
X₁LX₂YGIK

wherein:
   X₁ is E or G;
   X₂ is S or T;
with the proviso that if X₂ is T, the peptide or peptide analogue comprises no more than 25 amino acid residues;
   (ii) a peptide comprising or consisting of an amino acid sequence of the general formula:

(SEQ ID NO: 178)
Z₁Z₂SZ₃Z₄YGLR

wherein:
   Z₁ is D or G;
   Z₂ is I or G;
   Z₃ is V or L;
   Z₄ is V or A; and
   (iii) a peptide comprising or consists of an amino acid sequence selected from the group consisting of VDTYDGDISVVYGL (SEQ ID NO: 3) VDTYDGDISVVYG (SEQ ID NO: 6), VDTYDGDISVVY (SEQ ID NO: 10), VDTYDGDISVV (SEQ ID NO: 15), VDTYDGDISV (SEQ ID NO: 21) and VDTYDGDIS (SEQ ID NO: 28);
b) a polynucleotide encoding upon expression, the peptide of a);
c) a vector comprising the polynucleotide of b); or
d) a cell comprising the polynucleotide of b), or the vector of c);
for use in the treatment of an endocrine disease, a nutritional disease and/or a metabolic disease in a mammal.

In one aspect, the present disclosure relates to an agent comprising or consisting of a peptide or a peptide analogue comprising or consisting of an amino acid sequence of the general formula:

(SEQ ID NO: 177)
X₁LX₂YGIK

wherein:
   X₁ is E or G;
   X₂ is S or T;
with the proviso that if X₂ is T, the peptide or peptide analogue comprises no more than 25 amino acid residues;
for use in the treatment of an endocrine disease, a nutritional disease and/or a metabolic disease in a mammal.

In one aspect, the present disclosure relates to an agent comprising or consisting of a peptide or a peptide analogue comprising or consisting of an amino acid sequence of the general formula:

(SEQ ID NO: 178)
Z₁Z₂SZ₃Z₄YGLR

wherein:
   Z₁ is D or G;
   Z₂ is I or G;
   Z₃ is V or L;
   Z₄ is V or A;
for use in the treatment of an endocrine disease, a nutritional disease and/or a metabolic disease in a mammal.

In one aspect, the present disclosure relates to a peptide comprising or consists of an amino acid sequence selected from the group consisting of VDTYDGDISVVYGL (SEQ ID NO: 3) VDTYDGDISVVYG (SEQ ID NO: 6), VDTYDGDISVVY (SEQ ID NO: 10), VDTYDGDISVV (SEQ ID NO: 15), VDTYDGDISV (SEQ ID NO: 21) and VDTYDGDIS (SEQ ID NO: 28) for use in the treatment of an endocrine disease, a nutritional disease and/or a metabolic disease in a mammal.

In one embodiment, the present disclosure concerns a polynucleotide encoding upon expression, the peptide as described herein for use in the treatment of an endocrine disease, a nutritional disease and/or a metabolic disease in a mammal.

In one embodiment, the present disclosure concerns a vector comprising a polynucleotide as described herein for use in the treatment of an endocrine disease, a nutritional disease and/or a metabolic disease in a mammal.

In one embodiment, the present disclosure concerns a cell comprising a polynucleotide as described herein for use in the treatment of an endocrine disease, a nutritional disease and/or a metabolic disease in a mammal.

In one embodiment, the present disclosure concerns a cell comprising a vector as described herein for use in the treatment of an endocrine disease, a nutritional disease and/or a metabolic disease in a mammal.

In one aspect, the present disclosure relates to a composition for use in treatment of an endocrine disease, a nutritional disease and/or a metabolic disease, comprising an agent described herein. In one embodiment, said composition is a pharmaceutical composition.

In one embodiment, the agent further comprises a second active ingredient. Said second active ingredient may be selected from the group consisting of insulin, glucagon-like peptide-1 (GLP-1), biguanides, forskolin compounds, sulfonylurea, a dipeptidyl peptidase-4 (DPP4) inhibitor, an alpha-glucosidase inhibitor, a thiazolidinedione, a meglitidine and a sodium-glucose cotransporter-2 (SGLT2) inhibitor.

### Other Methods

In one aspect, the present disclosure concerns a method of treating an endocrine disease, a nutritional disease and/or a metabolic disease, the method comprising administering an agent, a composition, a polynucleotide, a vector or a cell as described herein, to a subject in need thereof.

In one aspect, the present disclosure concerns the use of an agent, a composition, a polynucleotide, a vector or a cell as described herein, for the manufacture of a medicament for use in treatment of an endocrine disease, a nutritional disease and/or a metabolic disease in a mammal.

In one aspect, the present disclosure concerns a polynucleotide encoding upon expression the peptide as described herein. In one aspect, the present disclosure concerns a vector comprising said polynucleotide encoding upon expression the peptide as described herein. In one aspect, the present disclosure concerns a cell comprising said polynucleotide or said vector encoding upon expression the peptide as described herein

In one aspect, the present disclosure concerns a method for increasing insulin secretion, the method comprising administering a therapeutically effective amount of a peptide or peptide analogue described herein, to an individual in need thereof. In one embodiment, said method is an in vitro method. In one aspect, the present disclosure concerns a method for increasing insulin secretion, the method comprising administering a therapeutically effective amount of an agent, a composition, a polynucleotide, a vector or a cell as described herein, to an individual in need thereof. In one embodiment, said method is an in vitro method.

In one aspect, the present disclosure concerns a method for decreasing blood glucose levels, the method comprising administering a therapeutically effective amount of a peptide or peptide analogue, an agent, a composition, a polynucleotide, a vector or a cell as described herein, to an individual in need thereof. In one embodiment, said method is an in vitro method. In one embodiment, insulin secretion is increased. In another embodiment, cellular uptake of glucose is increased. In yet another embodiment, insulin production is increased. In another embodiment glucagon production is decreased.

In one aspect, the present disclosure concerns a method, e.g. an in vitro method, for improving β-cell morphology, the method comprising administering a therapeutically effective amount of a peptide or peptide analogue, an agent, a composition, a polynucleotide, a vector or a cell as described herein, to an individual in need thereof.

In one aspect, the present disclosure concerns a method for improving β-cell viability, the method comprising administering a therapeutically effective amount of a peptide or peptide analogue, an agent, a composition, a polynucleotide, a vector or a cell as described herein, to an individual in need thereof.

In one aspect, the present disclosure concerns a method for delaying onset of diabetes and diabetes associated disorders and disease, the method comprising administering a therapeutically effective amount of a peptide or peptide analogue, an agent, a composition, a polynucleotide, a vector or a cell as described herein, to an individual in need thereof.

In one embodiment of the present disclosure, the agent may further comprise a detectable moiety. For example, a detectable moiety may comprise or consist of a radioisotope, such as a radioisotope selected from the group consisting of ^{99m}Tc, ¹¹¹In, ⁶⁷Ga, ⁶⁸Ga, ⁷²As, ⁸⁹Zr, ¹²³I and ²⁰¹TI. The binding moieties may thus be coupled to nanoparticles that have the capability of multi-imaging (for example, SPECT, PET, MRI, Optical, or Ultrasound). Alternatively, the detectable moiety may comprise or consist of a paramagnetic isotope, such as a paramagnetic isotope is selected from the group consisting of ¹⁵⁷Gd, ⁵⁵Mn, ¹⁶²Dy, ⁵²Cr and ⁵⁶Fe.

In the case that the agent comprises a detectable moiety, then the detectable moiety may be detectable by an imaging technique such as SPECT, PET, MRI, optical or ultrasound imaging.

In one aspect, the present disclosure concerns the use of an agent, a composition, a polynucleotide, a vector or a cell as described herein, for the preparation of a diagnostic composition for the diagnosis of a disease, disorder or damage of the pancreas in an individual.

### Items

1. An agent comprising a peptide or peptide analogue, wherein the peptide or peptide analogue comprises an amino acid sequence of the general formula:

(SEQ ID NO: 177)
X₁LX₂YGIK

wherein:
   X₁ is E or G;
   X₂ is S or T;
with the proviso that if X₂ is T, the peptide or peptide analogue comprises no more than 25 amino acid residues; and
with the proviso that if X₁ is E and X₂ is S, the peptide or peptide analogue comprises no more than 85 amino acid residues.
2. An agent comprising a peptide, wherein the peptide comprises an amino acid sequence of the general formula:

(SEQ ID NO: 178)
Z₁Z₂SZ₃Z₄YGLR

wherein:
   Z₁ is D or G;
   Z₂ is I or G;
   Z₃ is V or L;
   Z₄ is V or A.
3. The agent according to item 2, wherein the agent comprising a peptide, wherein the peptide comprises or consists of an amino acid sequence selected from the group consisting of DTYDGDISVVYGLR (SEQ ID NO: 4), TYDGDISVVYGLR (SEQ ID NO: 8), YDGDISVVYGLR (SEQ ID NO: 13), and DGDISVVYGLR (SEQ ID NO: 19). GDISVVYGLR (SEQ ID NO: 26), DISVVYGLR (SEQ ID NO: 34).
4. An agent comprising a peptide or peptide analogue comprising or consisting of the amino acid sequence DTYDGDISVVYGLR (SEQ ID NO: 4), TYDGDISVVYGLR (SEQ ID NO: 8), YDGDISVVYGLR (SEQ ID NO: 13), and DGDISVVYGLR (SEQ ID NO: 19). GDISVVYGLR (SEQ ID NO: 26), DISVVYGLR (SEQ ID NO: 34);
5. The agent according to any one of the preceding items, wherein the agent comprises non-naturally occurring, e.g. non-proteinogenic, amino acid residues.
6. The agent according to any one of the preceding items, wherein the agent is conjugated to a moiety.
7. The agent according to any one of the preceding items, wherein the moiety is selected from the group consisting of polyethylene glycol (PEG), monosaccharides, fluorophores, chromophores, radioactive compounds, and cell-penetrating peptides.
8. The agent according to any one of the preceding items, wherein the agent is further modified such as being glycosylated or by PEGylation, amidation, esterification, acylation, acetylation and/or alkylation.
9. The agent according to any one of the preceding items, wherein the agent comprises or consists of tandem repeats.
10. The agent according to any one of the preceding items, wherein the tandem repeats comprise or consist of the amino acid sequence of any one or more of the sequences as described in the preceding items.
11. The agent according to any of the preceding items, wherein the agent is fused to another polypeptide.
12. The agent according to any one of the preceding items, wherein the said polypeptide is selected from the group consisting of glutathione-S-transferase (GST) and protein A.
13. The agent according to any of the preceding items, wherein the agent is fused to a tag.
14. The agent according to any one of the preceding items, wherein the said tag is an oligo-histidine tag.
15. The agent according to any of the preceding items, wherein the agent is cyclic, such as wherein the peptide is cyclic.
16. The agent according to any of the preceding items, wherein the peptide or peptide analogue is capable of forming at least one intramolecular cysteine bridge, e.g. to form a cyclic or partially cyclic peptide.
17. The agent according to any of the preceding items, wherein the peptide or peptide analogue comprises or consists of an amino acid sequence selected from the group consisting of LAEIDSIELSYGIK (SEQ ID NO: 170), AEIDSIELSYGIK (SEQ ID NO: 171), EIDSIELSYGIK (SEQ ID NO: 172), IDSIELSYGIK (SEQ ID NO: 173), DSIELSYGIK (SEQ ID NO: 174), SIELSYGIK (SEQ ID NO: 175), IELSYGIK (SEQ ID NO: 148), KPLAEIDSIELTYGIK (SEQ ID NO: 176), or a variant or fragment thereof.
18. The agent according to any of the preceding items, wherein the peptide or peptide analogue comprises or consists of an amino acid sequence selected from the group consisting of KPLAEIDSIELSYGI (SEQ ID NO: 179), KPLAEIDSIELSYG (SEQ ID NO: 180), KPLAEIDSIELSY (SEQ ID NO: 181), KPLAEIDSIELS (SEQ ID NO: 182), KPLAEIDSIEL (SEQ ID NO: 183), KPLAEIDSIE (SEQ ID NO: 184), or a variant of fragment thereof.
19. The agent according to any of the preceding items, wherein the peptide or peptide analogue comprises or consists of the amino acid sequence LAEIDSIELSYGIK (SEQ ID NO: 170), or a variant or fragment thereof.
20. The agent according to any of the preceding items, wherein the peptide or peptide analogue comprises or consists of the amino acid sequence AEIDSIELSYGIK (SEQ ID NO: 171), or a variant or fragment thereof.
21. The agent according to any of the preceding items, wherein the peptide or peptide analogue comprises or consists of the amino acid sequence EIDSIELSYGIK (SEQ ID NO: 172), or a variant or fragment thereof.
22. The agent according to any of the preceding items, wherein the peptide or peptide analogue comprises or consists of the amino acid sequence IDSIELSYGIK (SEQ ID NO: 173), or a variant or fragment thereof.
23. The agent according to any of the preceding items, wherein the peptide or peptide analogue comprises or consists of the amino acid sequence DSIELSYGIK (SEQ ID NO: 174), or a variant or fragment thereof.
24. The agent according to any of the preceding items, wherein the peptide or peptide analogue comprises or consists of the amino acid sequence SIELSYGIK (SEQ ID NO: 175), or a variant or fragment thereof.
25. The agent according to any of the preceding items, wherein the peptide or peptide analogue comprises or consists of the amino acid sequence IELSYGIK (SEQ ID NO: 176), or a variant or fragment thereof.
26. The agent according to any one of the preceding items, wherein the variant comprises or consists of a sequence wherein any one amino acid has been altered for another proteinogenic or non-proteinogenic amino acid, with the proviso that no more than five amino acids are so altered.
27. The agent according to any one of the preceding items, wherein the variant comprises or consists of a sequence wherein no more than five amino acids are altered for another proteinogenic or non-proteinogenic amino acid, such as no more than 4 amino acids, such as no more than 3 amino acids, such as no more than 2 amino acids, such as no more than 1 amino acid is altered.
28. The agent according to any one of the preceding items, wherein one or more amino acids are conservatively substituted.
29. The agent according to any one of the preceding items, wherein the peptide or peptide analogue comprises or consists of one or more additional amino acids, inserted at the N-and/or C-terminus and/or internally within the sequence.
30. The agent according to any one of the preceding items, wherein the peptide or peptide analogue comprises 1 additional amino acid conjugated to either N- or C-terminal.
31. The agent according to any one of the preceding items, wherein the peptide or peptide analogue comprises or consists of one proline inserted at the N-terminus.
32. The agent according to any of the preceding items, wherein the agent comprises no more than 85, such as no more than 80, such as no more than 75, such as no more than 70, such as no more than 65, such as no more than 60, such as nor more than 55, such as no more than 50, such as no more than 55, such as no more than 40 amino acids, such as no more than 35, such as no more than 30, such as no more than 28, such as no more than 26, such as no more than 24, such as no more than 22, such as no more than 20, such as no more than 19, such as no more than 18, such as no more than 17, such as no more than 16, such as no more than 15, such as no more than 14, such as no more than 13, such as no more than 12, such as no more than 11, such as no more than 10 amino acids.
33. The agent according to any one of the preceding items, wherein the agent comprises at least 2 additional amino acids, such as at least 3, such as at least 4, such as at least 5, such as at least 6, such as at least 7, such as at least 8, such as at least 9, such as at least 10, such as at least 15 or such as at least 20 amino acids conjugated to the N- or C-terminus of the peptide or the peptide analogue.
34. The agent according to any of the preceding items, wherein the agent further comprises a detectable moiety.
35. The agent according to any of the preceding items, wherein the detectable moiety comprises or consists of a radioisotope.
36. The agent according to any of the preceding items, wherein the radioisotope is selected from the group consisting of ^{99m}Tc, ¹¹¹In, ⁶⁷Ga, ⁶⁸Ga, ⁷²As, ⁸⁹Zr, ¹²³I and ²⁰¹TI.
37. The agent according to any of the preceding items, wherein the detectable moiety is detectable by an imaging technique such as SPECT, PET, MRI, optical or ultrasound imaging.
38. Use of the agent of any of the preceding items, for the preparation of a diagnostic composition for the diagnosis of a disease, disorder or damage of the pancreas in an individual.
39. A polynucleotide encoding upon expression, a peptide or peptide analogue according to any one of the preceding claims.
40. A vector comprising a polynucleotide according to claim 39.
41. A cell comprising a polynucleotide according to claim 39, or a vector according to claim 40.
42. A composition comprising the agent according to any of the preceding items.
43. The composition according to any one of the preceding items, wherein the composition is a pharmaceutical composition.
44. The agent according to claims 1-37, the polynucleotide according to claim 39, the vector according to claim 40, the cell according to claim 41 or the composition according to claims 42-43, for use as a medicament.
45. An agent selected from the group consisting of:
   a) a peptide selected from the group consisting of
      (i) a peptide comprising or consisting of an amino acid sequence of the general formula:

(SEQ ID NO: 177)
X₁LX₂YGIK

wherein:
   X₁ is E or G;
   X₂ is S or T;
   with the proviso that if X₂ is T, the peptide or peptide analogue comprises no more than 25 amino acid residues;
      (ii) a peptide comprising or consisting of an amino acid sequence of the general formula:

(SEQ ID NO: 178)
Z₁Z₂SZ₃Z₄YGLR

wherein:
   Z₁ is D or G;
   Z₂ is I or G;
   Z₃ is V or L;
   Z₄ is V or A; or
      (iii) a peptide comprising or consists of an amino acid sequence selected from the group consisting of VDTYDGDISVVYGL (SEQ ID NO: 3) VDTYDGDISVVYG (SEQ ID NO: 6), VDTYDGDISVVY (SEQ ID NO: 10), VDTYDGDISVV (SEQ ID NO: 15), VDTYDGDISV (SEQ ID NO: 21) and VDTYDGDIS (SEQ ID NO: 28);
b) a polynucleotide encoding upon expression, the peptide of a);
c) a vector comprising the polynucleotide of b); or
d) a cell comprising the polynucleotide of b), or the vector of c);
for use in the treatment of an endocrine disease, a nutritional disease and/or a metabolic disease in a mammal.
46. The agent or the composition for use according to any one of the preceding items, wherein the peptide is selected from the group consisting of SEQ ID NO: 141, 142, 143, 144, 145, 146, 147, 148, 149, 150, 151, 152, 153, 154, 155 and 156.
47. The agent or the composition for use according to any one of the preceding items, wherein the peptide is selected from the group consisting of SEQ ID NO: 1, 136, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 67, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 135, 137, 138, 139, 157, 158, 159, 160, 161, 167, 168, 169, 170, 171, 172, 173, 174, 175, 176, 177, 178, 179, 180, 181, 182, 183 and 184.
48. The agent or the composition for use according to any one of the preceding items, wherein said agent comprises a second or further active ingredient.
49. The agent or the composition for use according to item 48, wherein the second or further active ingredient is selected from the group consisting of insulin, glucagon-like peptide-1 (GLP-1), sulfonylurea, a dipeptidyl peptidase-4 (DPP4) inhibitor, an alpha-glucosidase inhibitor, a thiazolidinedione, a meglitidine and a sodium-glucose cotransporter-2 (SGLT2) inhibitor.
50. The agent or the composition according to any of the preceding items for use in the treatment of an endocrine disease, a nutritional disease and/or a metabolic disease in a mammal.
51. The agent or the composition for use according to item 50, wherein the mammal is a human.
52. The agent or the composition for use according to any one of the preceding items, wherein the endocrine disease, nutritional disease and/or metabolic disease are selected from the group consisting of diabetes mellitus, type 1 diabetes mellitus, type 2 diabetes mellitus, malnutrition-related diabetes mellitus, disorders of glucose regulation and pancreatic internal secretion, insulin resistance syndrome, impaired glucose tolerance, hyperglycemia, hyperinsulinemia, and any combinations thereof.
53. The agent or the composition for use according to any one of the preceding items, wherein the endocrine disease, nutritional disease and/or metabolic disease are selected from the group consisting of diabetes mellitus, disorders of the thyroid gland, disorders of glucose regulation and pancreatic internal secretion, disorders of endocrine glands, malnutrition, nutritional deficiencies, obesity, hyperalimentation, and metabolic disorders.
54. The agent or the composition for use according to any one of the preceding items, wherein the diabetes mellitus is selected from the group consisting of type 1 diabetes mellitus, type 2 diabetes mellitus, malnutrition-related diabetes mellitus, specified diabetes mellitus, and unspecified diabetes mellitus.
55. The agent or the composition for use according to any one of the preceding items, wherein the disorder of glucose regulation and pancreatic internal secretion is selected from the group consisting of nondiabetic hypoglycaemic coma and disorders of pancreatic internal secretion.
56. The agent or the composition for use according to any one of the preceding items, wherein the disorder of obesity and hyperalimentation is selected from the group consisting of localized adiposity, hyperalimentation, and sequelae of hyperalimentation.
57. The agent or the composition for use according to any one of the preceding items, wherein the disorder of nutritional deficiencies is selected from the group consisting of disorders of aromatic amino-acid metabolism, disorders of branched-chain amino-acid metabolism and fatty-acid metabolism, disorders of amino-acid metabolism, lactose intolerance, disorders of carbohydrate metabolism, disorders of sphingolipid metabolism, disorders of lipid storage disorders, disorders of glycosaminoglycan metabolism, disorders of glycoprotein metabolism, disorders of lipoprotein metabolism, lipiemias, disorders of purine and pyrimidine metabolism, disorders of porphyrin and bilirubin metabolism, disorders of mineral metabolism, cystic fibrosis, amyloidosis, volume depletion, disorders of fluid, electrolyte and acid-base balance, and postprocedural endocrine and metabolic disorders.
58. A method of treating an endocrine disease, a nutritional disease and/or a metabolic disease, the method comprising administering an agent according to any one of the preceding items to a subject in need thereof.
59. Use of an agent according to any one of the preceding items for the manufacture of a medicament for use in treatment of an endocrine disease, a nutritional disease and/or a metabolic disease in a mammal.
60. A method for delaying onset of diabetes and diabetes associated disorders and diseases, the method comprising administering a therapeutically effective amount of the agent as defined in any one of the preceding items, to an individual in need thereof.
61. A method for decreasing blood glucose levels, the method comprising administering a therapeutically effective amount of an agent of any one of the preceding items, to an individual in need thereof.
62. The method according to item 61, wherein insulin secretion is increased.
63. The method according to item 61, wherein cellular uptake of glucose is increased.
64. The method according to item 61, wherein the insulin production is increased.
65. The method according to item 61, wherein the glucagon production is decreased.
66. A method for improving beta cell viability, the method comprising administering a therapeutically effective amount of an agent of any one of the preceding items, to an individual in need thereof.
67. A method for improving beta cell morphology, the method comprising administering a therapeutically effective amount of an agent of any one of the preceding items, to an individual in need thereof.
68. A method for stabilising or improving viability and/or morphology of pancreatic islets, the method comprising administering a therapeutically effective amount of an agent of any one of the preceding items, to an individual in need thereof.

### EXAMPLES

The disclosure is further illustrated by the following examples, which however should not be construed as being limiting for the disclosure. These examples demonstrate that exemplary peptides of the present disclosure stimulate β-cell proliferation, and have the ability to protect and rescue β-cells from apoptosis induced by glucotoxic conditions. It is also demonstrated that the exemplary peptides have the ability to stimulate insulin secretion from rat β-cells as well as isolated mouse pancreatic islets, where the peptides also are demonstrated to reduce glucagon levels. Furthermore, the examples demonstrate that the peptides reduce plasma glucose levels in vivo in a glucose tolerance test and that the peptides delay onset of type 1 diabetes in BB lyp/lyp rats

### Example 1: Peptide Design

The novel peptides were designed following rational structure activity investigations. For FOL-005 (SEQ ID NO: 1) the peptides were designed around the RGD site but mutated in order to generate different structures that potentially could interact with different integrins. A sequence similar to FOL-005 was identified in the third fibronectin type III repeat domain (TNfn3) in tenascin-C and found to be reasonably similar to the mutated RGD site of FOL-005. A peptide was designed from this sequence denoted FOL-014. The X-ray crystal structure of the tenascin-3 TNfn3 domain (PDB code 1TEN, Leahy et al. (1992) Science 258(5084):987-91) was analysed. The FOL-014 (SEQ ID NO: 136) sequence span the beta-turn before and the entire 3rd beta sheet. FOL-014 variants were designed to allow for structural modification and stabilization of the 3-dimensional molecular structure. Specifically, the peptides variants covered the beta-turn region with exposed side chains and some cyclized variants to maintain geometry.

All peptides were synthesized by solid phase peptide synthesis using several peptide manufacturers. Mainly, the peptide variants have been provided by Biopeptide Inc., California.

### Example 2: FOL-005 and FOL-014 Induced Proliferation of INS-1 Cells

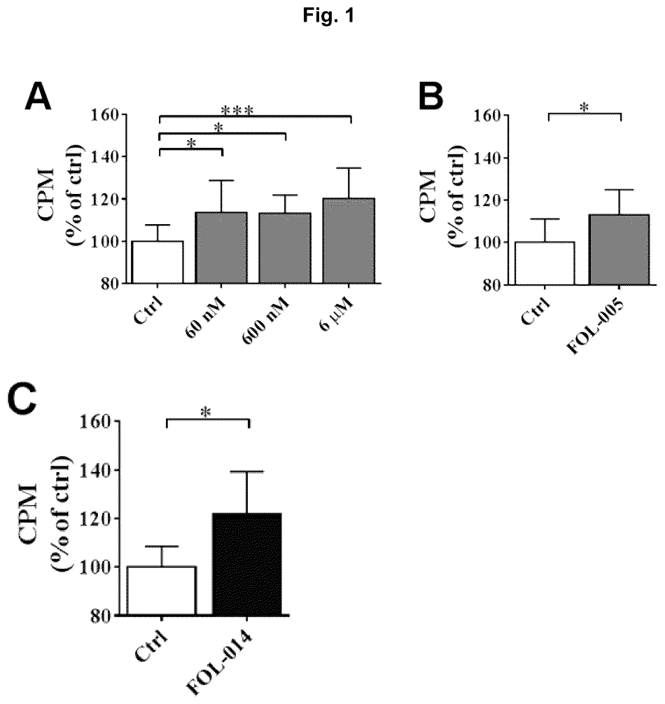

To investigate if FOL-005 and FOL-014 could induce proliferation of β-cells we used INS-1 cells. Rat INS-1 cells were seeded in 96-well plates in RPMI medium with supplement and after 2 hours the medium was changed to RPMI without supplement. During the proliferation experiment the cells were incubated at different test conditions (FOL-005, FOL-014, coated or in solution, 48 h incubation) and during the last 20 hours of culture period the cells were pulsed with 1 μ Ci/well of [methyl-3H] thymidine. The cells were then harvested onto glass fiber filters using a FilterMate harvester. The filters were air dried, and the bound radioactivity was measured using a liquid scintillation counter. To study whether FOL-005 influenced β-cell proliferation, INS-1 cells were treated with increasing amounts of soluble FOL-005 (0.06-6 μM) during 48 hours and proliferation was measured with radiolabeled thymidine incorporation into newly synthesized DNA. FOL-005 stimulated INS-1 cell proliferation (FIG. 1A). Wells coated with either FOL-005 or FOL-014 and later blocked with bovine serum albumin (BSA) before addition of INS-1 cells also stimulated proliferation compared to control (ctrl) coated wells (FIG. 1B-C).

This demonstrated that FOL-005 and FOL-014 interacted with β-cells and induced proliferation.

### Example 3: FOL-005 Protected β-Cells from Glucotoxicity

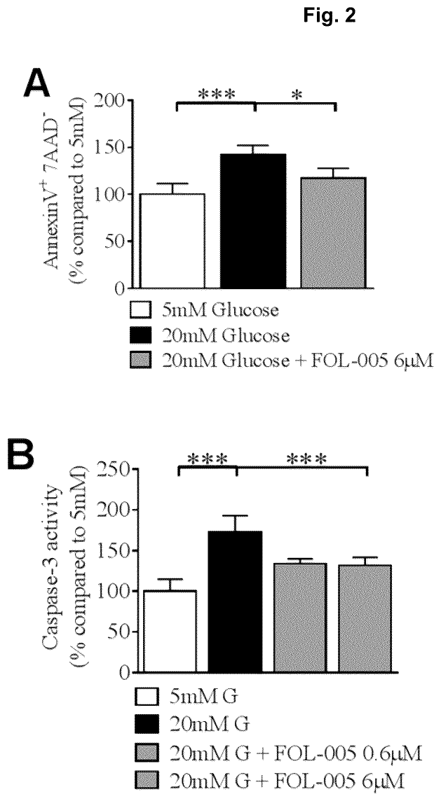

Since glucotoxicity in pancreatic β-cells is a well-established process in type 2 diabetes we next investigated the protective effects of FOL-005 on β-cells during glucotoxic conditions. First we confirmed that 20 mM glucose induced cell apoptosis in INS cells after 48 h of exposure. High glucose (20 mM) containing RPMI medium induced more Annexin V positive cells and more caspase-3 activity in INS cells compared to cells incubated with medium containing 5 mM glucose (FIG. 2A-B). Exposure of INS-1 cells to 20 mM of glucose at the same time as FOL-005 decreased cell apoptosis as detected both by Annexin V staining and by caspase-3 activity (FIG. 2A-B). The rate of apoptosis in INS-1 cells was measured with either Caspase-3 Assay Kit or stained with Annexin V Apoptosis Detection Kit with 7-AAD. Caspase-3 activity was measured with fluorescence at an excitation wavelength of 380 nm and an emission wavelength of 440 nm. Caspase-3 activity was then normalized to protein concentration in each well. Measurements of

Annexin V stained cells were performed using a CyAn ADP flow cytometer and analyzed with Summit V4.3 software.

In conclusion, it is well known that glucotoxicity induces β-cell apoptosis, however in the presence of FOL-005 glucotoxicity-induced apoptosis was diminished.

### Example 4: FOL-005 Induced Insulin Secretion from INS-1 Cells

To investigate the stimulatory effect of FOL-005 on insulin secretion, INS-1 β-cells were used in the following experiments. Cells were seeded overnight in cRPMI and then washed with PBS before pre-incubation for 60 min at 37° C. in Krebs-Ringer bicarbonate buffer (KRB), pH 7.4, supplemented with 10 mM HEPES, 0.1% bovine serum albumin. After pre-incubation, the buffer was changed and the INS-1 cells were incubated at different test conditions (0 mM, 5 mM or 20 mM glucose) and stimulated with peptide FOL-005 or FOL-015 (SEQ ID NO: 158) or left untreated during 60 min at 37° C. Immediately after incubation, an aliquot of the buffer was removed and frozen for subsequent assay of insulin with an insulin radioimmunoassay kit.

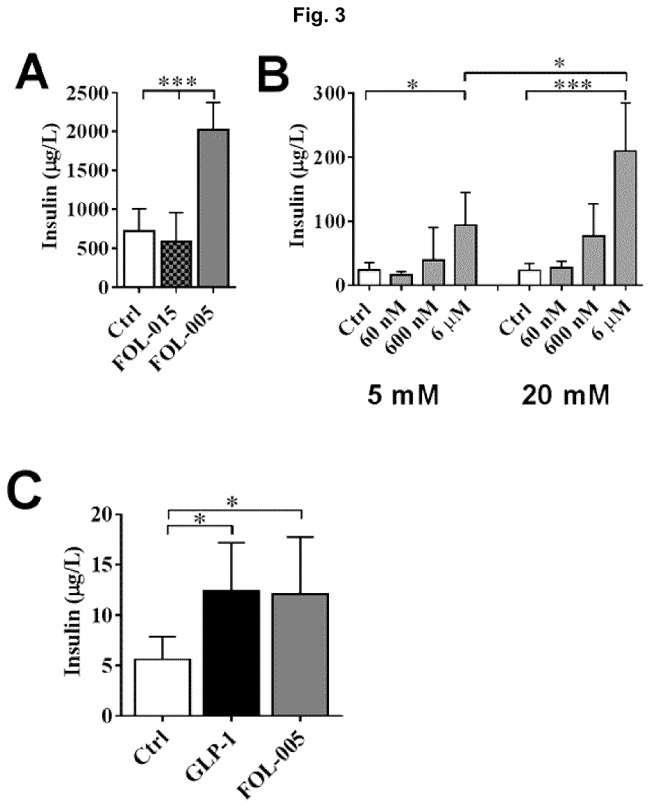

The results demonstrated that β-cells stimulated with FOL-005 peptide secreted more insulin compared to unstimulated control cells or to cells stimulated with the FOL-015 control peptide (FIG. 3A) under conditions without glucose. INS-1 β-cells subjected to glucose (5 mM or 20 mM) responded with insulin secretion after FOL-005 peptide (6 μM) stimulation (FIG. 3B). INS-1 cells stimulated with 6 μM FOL-005 peptide in the presence of 20 mM glucose responded with more insulin secretion compared to FOL-005 stimulated cells incubated with 5 mM glucose (FIG. 3B).

### Example 5: FOL-005 Induced Insulin Secretion from Mouse Pancreatic Islets

Mouse pancreatic islets were isolated from 8-week old C57BL/6J male mice (Taconic). Mice were sacrificed by an overdose of isoflurane and cervical dislocation. 3 ml of 0.9 U/ml collagenase P was injected into the pancreatic duct to inflate the pancreas. The Pancreas was then removed and collagen digested for 19 min at 37° C. The samples Were vigorously shaken to disrupt the tissue. The digest was transferred into ice cold Hank's Balanced Salt Solution (HBSS) with Ca²⁺ and Mg²⁺. The suspension was allowed to sit for 10 min to allow the islet to sink, and the islets were washed in fresh HBSS four times. The islets were then hand-picked and sorted according to size. Islets (n=3 per well in a 96 well plate) were pre-incubated in KRB buffer during 10 min 37° C., pH 7.4, supplemented with 10 mM HEPES, 0.1% bovine serum albumin. After pre-incubation, the buffer was changed and islets were incubated at different test conditions in new KRB buffer with 0.1% bovine serum albumin (non-treated ctrl, FOL-005 peptide, or GLP-1) for 60 min at 37° C. Immediately after incubation, an aliquot of the buffer was removed and frozen for subsequent assay of insulin.

The results demonstrated that isolated mouse pancreatic islets stimulated with GLP-1 (100 nM) or FOL-005 (6 μM) secreted more insulin compared to unstimulated control islets (FIG. 3C).

### Example 6: FOL-014 Induced Insulin Secretion from INS-1 Cells

INS-1 β-cells were used to investigate the stimulatory effect of FOL-014 on insulin secretion. Cells were seeded overnight and then washed with PBS before pre-incubation for 60 min at 37° C. in Krebs-Ringer bicarbonate buffer (KRB), pH 7.4, supplemented with 10 mM HEPES, 0.1% bovine serum albumin. After pre-incubation, the buffer was changed and the INS-1 cells were incubated in new KRB buffer supplemented with 10 mM HEPES, 0.1% bovine serum albumin and stimulated with peptide FOL-014 or left untreated during 60 min at 37° C. Immediately after incubation, an aliquot of the buffer was removed and frozen for subsequent assay of insulin.

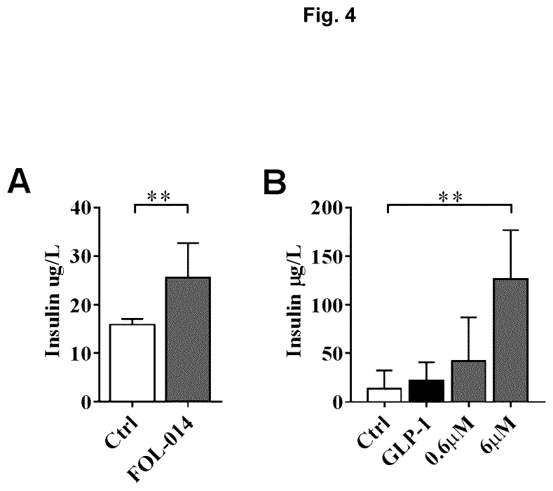

The results demonstrated that β-cells stimulated with FOL-014 peptide secreted more insulin compared to unstimulated control cells (FIG. 4A).

### Example 7: FOL-014 Induced Insulin Secretion from Mouse Pancreatic Islets

Mouse pancreatic islets were isolated from 8-week old C57BL/6J male mice as described under example 5. The islets were then hand-picked and sorted according to size. Islets (n=5 per well in a 96 well plate) were pre-incubated in 200 μl KRB buffer during 10 min 37° C., pH 7.4, supplemented with 10 mM HEPES, 0.1% bovine serum albumin. Following pre-incubation, the buffer was changed and islets were incubated in different test conditions in new KRB buffer with 0.1% bovine serum albumin (non-treated ctrl, FOL-014 peptide, and GLP-1) for 60 min at 37° C. Immediately after incubation, an aliquot of the buffer was removed and frozen for subsequent assay of insulin.

The result show that mouse pancreatic islets stimulated with FOL-014 (6 μM) secreted more insulin compared to unstimulated control islets (FIG. 4B). GLP-1 (100 nM) or FOL-014 (0.6 μM) did not affect insulin secretion (FIG. 4B).

### Examples 8-11; 20-21: Stimulation of Insulin Secretion from INS-1 Cell Lines by FOL-014, FOL-005 and Related Peptides

Materials and methods: Rat INS-1 β-cells (passages 60-70) were cultured at 37° C. and 5% CO₂ in cRPMI media (RPMI 1640 supplemented with 10% fetal bovine serum, 50 IU/mL penicillin, 50 mg/L streptomycin, 10 mM HEPES, 2 mM L-glutamine, 1 mM sodium pyruvate, and 50 μM beta-mercaptoethanol) unless otherwise stated. INS-1 cells were seeded in 96-well plates (2×10³ cells/well) in cRPMI medium and following overnight incubation, the cells were washed in PBS before pre-incubation for 120 min at 37° C. in Krebs-Ringer bicarbonate buffer, pH 7.4, supplemented with 10 mM HEPES, 0.1% bovine serum albumin and 2.8 mM glucose. Following pre-incubation, the buffer was exchanged with fresh Krebs-Ringer buffer as described above and supplemented with specific glucose concentrations and peptides for the individual experiments as described below. Immediately after 60 minutes incubation at 37° C., an aliquot of the buffer was removed and frozen for subsequent insulin ELIZA assay.

### Example 8. FOL-014 Induced Insulin Secretion is Dose-Dependent in a Non-Linear Manner

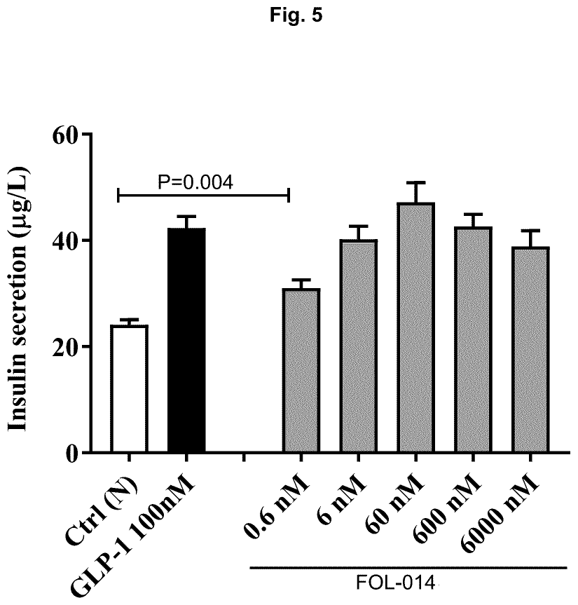

Insulin release from INS-1 cells were measured following exposure to increasing concentrations of FOL-014 and compared with the stimulatory effect of GLP-1 and untreated control during high glucose concentration (16.7 mM). All concentrations of FOL-014 tested elicited significantly higher insulin release as compared with the untreated control. At 6 nM or higher, FOL-014 triggered insulin release within the same range as 100 nM GLP-1. At concentrations ranging from 0.6-60 nM, insulin secretion increased in a linear fashion in relation to increasing FOL-014 concentrations. Exposure to FOL-014 concentrations ≥600 nM did not increase the insulin secretion (FIG. 5).

The results demonstrated that FOL-014 significantly increased insulin secretion from INS-1 β-cells in vitro in a non-linear dose dependent fashion.

### Example 9. The Capacity of FOL-014 to Induce Insulin Secretion is Glucose Dependent

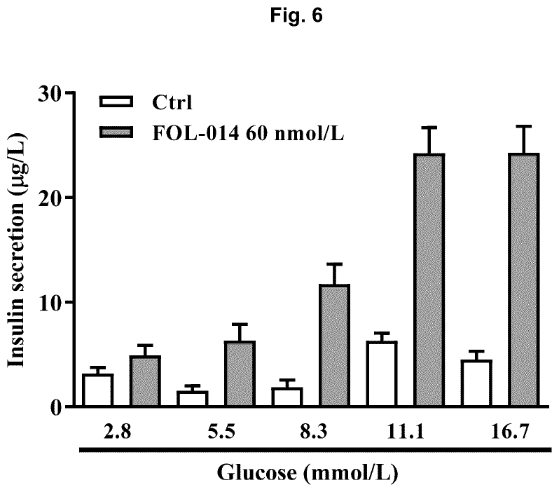

Insulin release from INS-1 cells was measured following exposure to 60 nM FOL-014 at increasing concentrations of glucose. In untreated control samples, elevated glucose concentrations increased the insulin secretion at 11.1 mM glucose or higher. In the presence of FOL-014, insulin secretion increased significantly in a glucose dependent fashion already from 5.5 mM glucose. (FIG. 6).

The results demonstrated that the presence of FOL-014 significantly increased insulin secretion from INS-1 β-cells in vitro in a glucose concentration dependent fashion and that FOL-014 was effective also at marginally elevated glucose levels.

### Example 10. FOL-014 or FOL-005 in Combination with GLP-1 Increased Insulin Secretion as Compared with Either Peptide Alone

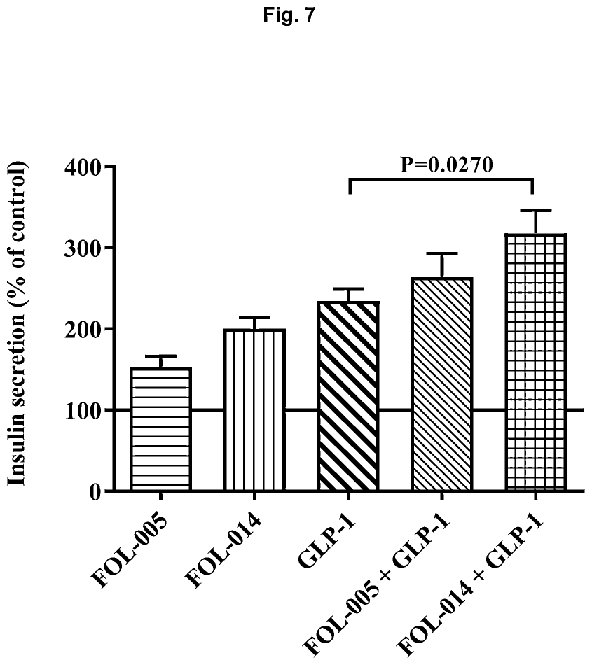

Insulin secretion from INS-1 cells was measured following exposure to FOL-005, FOL-014, GLP-1 or combinations of those, expressed as percentage of untreated control. The combined effect of GLP-1 and FOL-014 resulted in a significantly higher insulin release than GLP-1 or FOL-014 alone. The additive effect of the combination of FOL-005 and GLP-1 was less pronounced, but did however increase the insulin secretion as compared with GLP-1 alone. The experiments were performed in the presence of 16.7 mM glucose (FIG. 7).

The results demonstrated that the combination of GLP-1 and FOL-014 could further potentiate the insulin secretion from INS-1 cells in vitro as compared with each peptide alone. Furthermore, the combination of FOL-005 and GLP-1 tangentially increased insulin secretion.

### Example 11. The Ability of Novel Peptide Analogues to Induce Insulin Secretion in Pancreatic β-Cell-Lines was Investigated

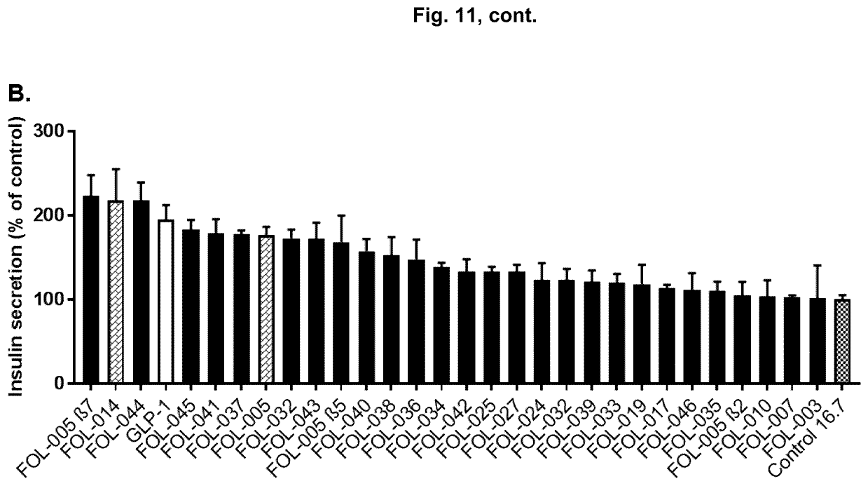

Novel peptide analogues, derived from either FOL-005 or FOL-014 were tested concerning their ability to induce insulin secretion in two separate INS-1 cell lines in the presence of 16.7 mM glucose. FOL-005, FOL-014 and GLP-1 as well as a high glucose (16.7 mM) and a low glucose (2.8 mM) control (not shown) was included in each experiment and the peptide concentration was 100 nM. In order to correct for the variance between experiments, all values were normalized to, and expressed as percentage of the average value of the high glucose control in the individual experiments. The analogues were subsequently ranked according to performance (FIGS. 11A and 11B). Peptide analogues eliciting an insulin response below the high glucose control average value were considered non-functional and were hence excluded (not shown).

The results demonstrated the capacity of several novel peptide analogues to enhance insulin secretion from INS-1 β-cells in vitro.

### Example 12. FOL-014 Increase Insulin Secretion from Mouse-Derived Pancreatic Islets

Twelve-week-old male C57/bl6 mice were euthanized with isoflurane and cervical dislocation. After clamping the hepatic ducts, 3 ml of 0.9 U/ml collagenase P was injected into the bile duct to inflate the pancreas. The pancreas was then removed and digested for 19 min at 37° C. The samples were vigorously shaken to disrupt the tissue. The digest was quickly transferred into ice cold Hank's Balanced Salts Solution with Ca²⁺ and Mg²⁺. The suspension was allowed to sit for 8 min to allow the islet to sink, and the islets were washed in the same manner four times. The islets were then handpicked and sorted according to size.

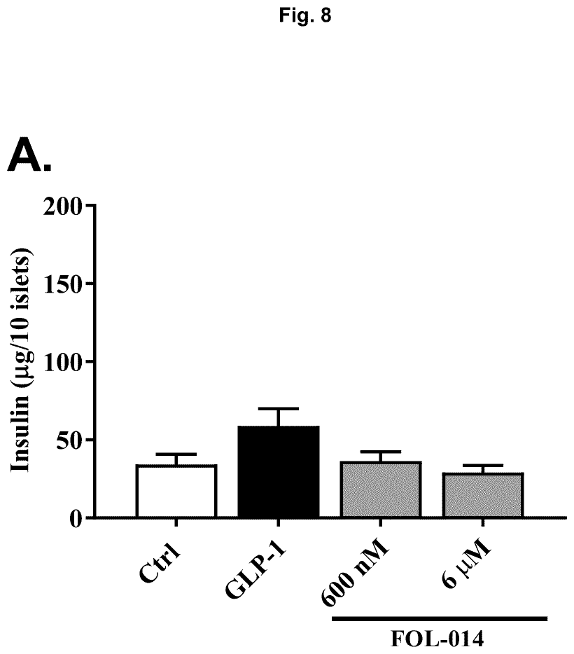

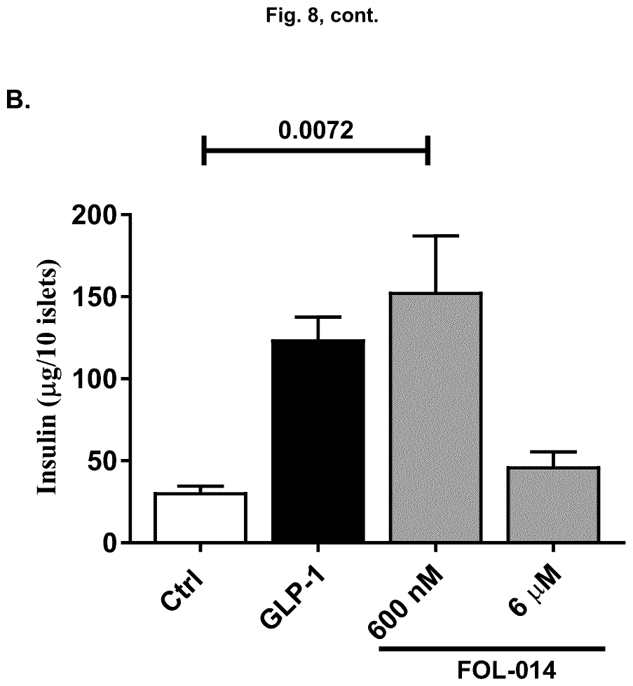

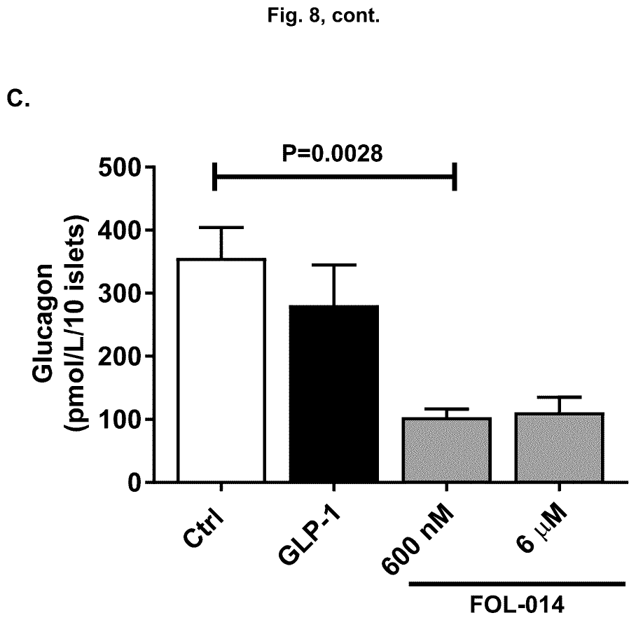

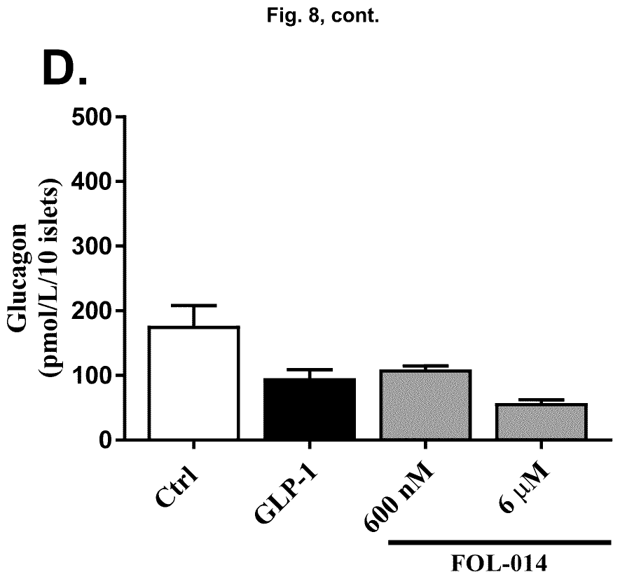

Freshly isolated islets were seeded in groups of 5 in a 96-well plate and preincubated for 1 h at 37° C. in a Krebs-Ringer bicarbonate buffer (pH 7.4). The islets were incubated for 1 h at 37° C. in Krebs-Ringer buffered solution supplemented with 0.6 or 6 μM FOL-014 or 100 nM GLP-1 or left unsupplemented for control. Immediately after incubation, the medium was removed for assays of insulin and glucagon using Mercodia's ELISA kits. The effect of FOL-014 on insulin (FIGS. 8A and B) and glucagon (FIGS. 8C and D) secretion from isolated mouse islets was measured in the presence of low glucose (2.8 mM; FIGS. 8A and C) or high glucose (16.7 mM; FIGS. 8B and D) concentrations. A significant effect of FOL-014 was observed in the presence of high glucose for insulin and in the presence of both high and low glucose for glucagon. The effect of FOL-014 differed from that of GLP-1, which enhanced insulin secretion also in low glucose samples but failed to inhibit glucagon secretion in low glucose conditions.

The results demonstrated that FOL-014 enhanced insulin secretion and inhibited glucagon secretion in pancreatic islets.

### Example 13. FOL-014 Reduced Plasma Glucose Levels in an Intraperitoneal Glucose Tolerance Test (IPGTT) in Mice

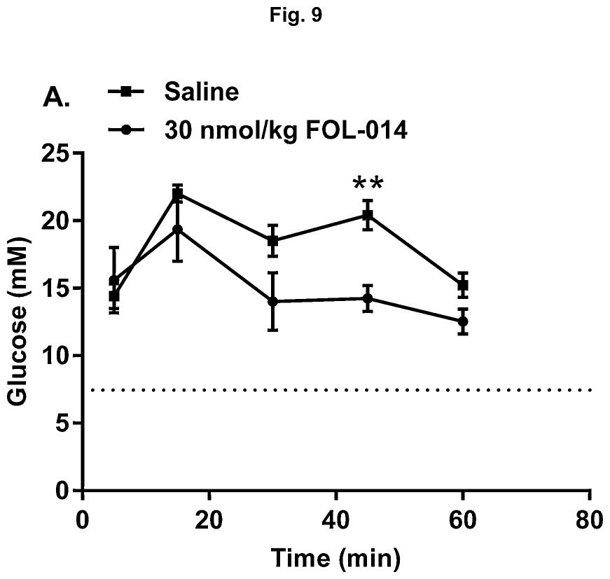

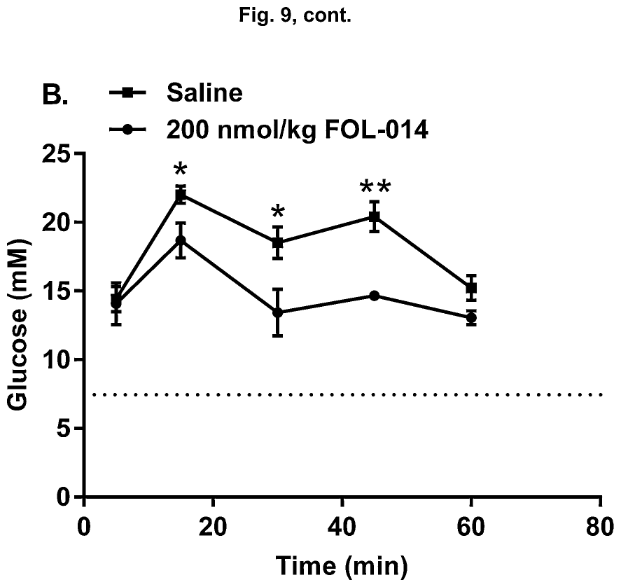

Whole blood was collected for glucose and insulin measurements from 10-week-old wild type maleC57bl/6 mice. After a 4 hour fast, the mice were divided into three groups and given an intraperitoneal injection (ip) of either saline, 30 nmol/kg peptide (FIG. 9A) or 200 nmol/kg peptide (FIG. 9B). 15 min after the FOL-014 or saline (control) injections, the mice were administered 2 g of glucose/kg ip. Blood glucose concentrations were measured at 5, 15, 30, 45 and 60 minutes after the glucose injection. Statistical calculations were performed using student's t-test. FOL-014 dosed at 200 nmol/kg significantly lowered the plasma glucose levels as compared to the control when measured as area under the curve. In addition, the difference was significant at 15, 30 and 45 minutes. At the 30 nmol/kg dose, FOL-014 lowered the plasma glucose levels with a significant effect at 45 minutes after the glucose injection.

The results demonstrated that FOL-014 could lower plasma glucose levels in a glucose tolerance test performed on healthy wild type mice.

### Example 14. FOL-014 Delayed Onset of Type 1 Diabetes in BB lyp/lyp Rats

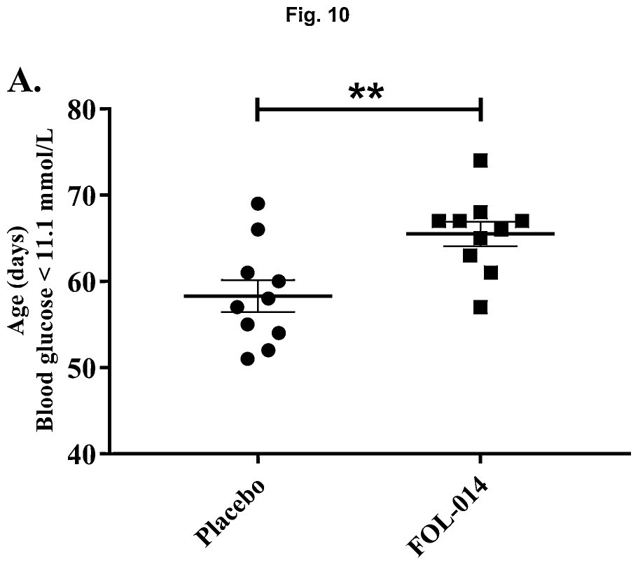

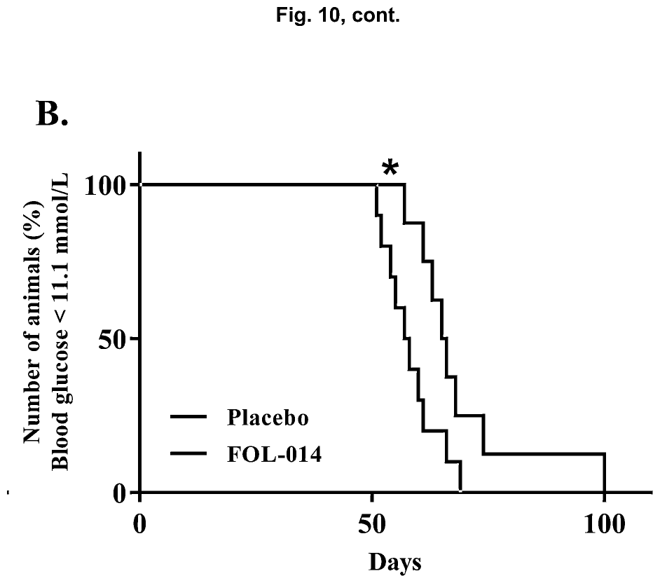

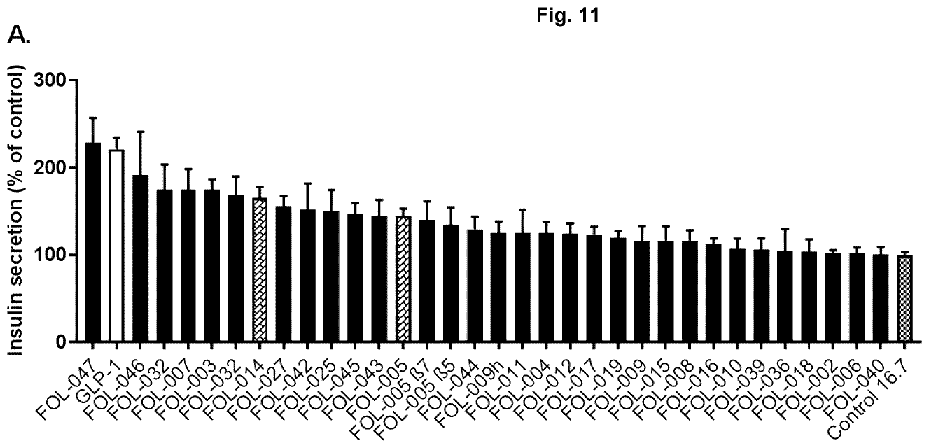

BB lyp/lyp rats were randomized for placebo (sodium chloride, 9 mg/ml) or FOL-014 treatment 3 times/week from day 40 until onset of type 1 diabetes, defined as plasma glucose levels ≥11.1 mM. The dose of 100 nmol/kg FOL-014 peptide in saline or placebo (saline) was administered subcutaneously and the animals were terminated immediately upon exceeding critical plasma glucose levels. The difference between FOL-014 treated animals and animals receiving placebo treatment was significant both when expressed as average age for onset of type 1 diabetes (FIG. 10A) and when described as percentage of animals developing type 1 diabetes per day (FIG. 10B).

The results demonstrated that FOL-014 treatment significantly delayed the onset of type-1 diabetes in BB lyp/lyp rats.

### Example 15. FOL-005 and FOL-014 Displayed Organ Specific Distribution Patterns in Mice

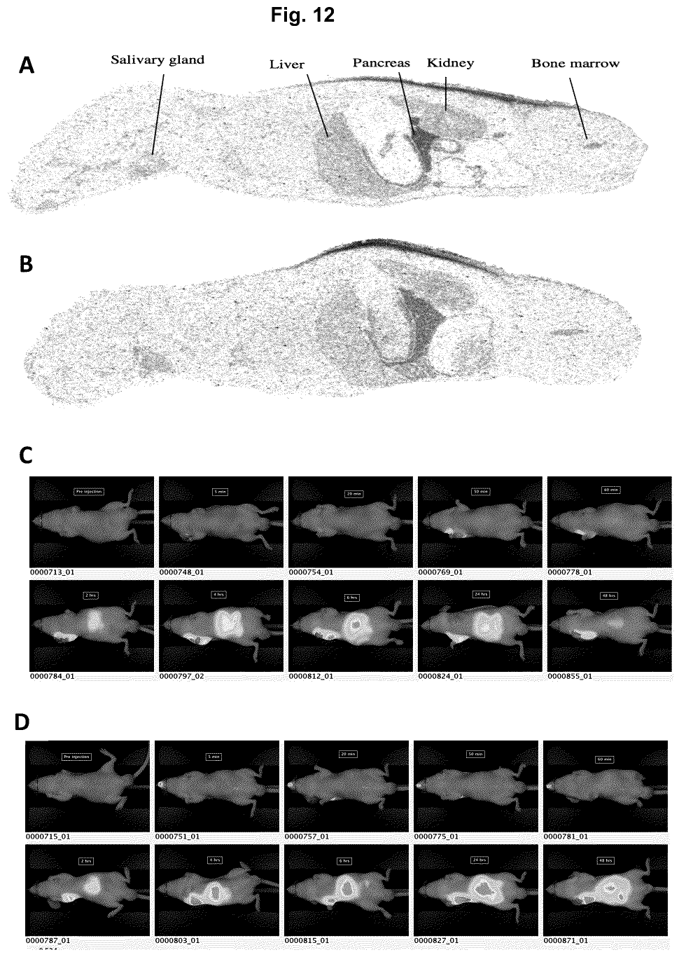

C57BI/6 mice were injected subcutaneously with H³ labelled FOL-005 and euthanized at 1 h (FIG. 12A) or 2 h (FIG. 12B) after injection. Following whole body sectioning the distribution of the labelled peptide was visualised. Strong binding was evident in pancreas and at the site of injection. Using Pearl Trilogy Small Animal Imaging System, in vivo bio-distribution and tissue localization of two Cy7.5 labelled peptides, FOL-005 (FIG. 12C) and FOL-014 (FIG. 12D) in NMRI nude mice via subcutaneous injection was investigated. High accumulation of the peptide was evident in the pancreatic tissue area. The same distribution pattern was found after i.v. administrations (not shown). The dose of each peptide was 10 nmol per mouse. The mice were imaged before injection, at 5 min, 20 min, 50 min, 60 min, 2 hrs, 4 hrs, 6 hrs, 24 hrs and 48 hrs post administration of labelled peptide.

### Example 16. Tissue Specific Imaging for Diagnostic Use

Agents prepared as defined herein above are labelled by conjugation to suitable imaging probe or moiety, using methods known by those of skill in the art. The conjugated peptide-probe agents are subsequently administered to a subject and biodistribution is subsequently monitored e.g. up to 48 h after administration. The conjugated agent is thus used as a diagnostic or prognostic tool for investigation of pancreatic status. As such, the conjugated agents are suitable for detecting, diagnosing, or monitoring disease, disease processes and progression, susceptibility, as well as to determine efficacy of a treatment. The agents are particularly suited for monitoring the diabetic status of a subject. The conjugated agents are also used for monitoring and/or predicting risk of developing a disease, specifically diabetes. The test is used alone or in combination with other tests known by those of skill in the art, such as blood tests, genetic testing, urine test, and biopsies.

### Example 17: Sequence Overview

Example 17: Sequence overview

| SEQ | | |
|---|---|---|
| ID | | |
| NO | Sequence | Notes |
| 1 | VDTYDGDISVVYGLR | FOL-005 |
| 2 | VDTYDGDISVVYGLS | |
| 3 | VDTYDGDISVVYGL | FOL-025 |
| 4 | DTYDGDISVVYGLR | FOL-061 |
| 5 | TYDGDISVVYGLRS | |
| 6 | VDTYDGDISVVYG | FOL-024 |
| 7 | DTYDGDISVVYGL | |
| 8 | TYDGDISVVYGLR | |
| 9 | YDGDISVVYGLRS | |
| 10 | VDTYDGDISVVY | |
| 11 | DTYDGDISVVYG | |
| 12 | TYDGDISVVYGL | |
| 13 | YDGDISVVYGLR | |
| 14 | DGDISVVYGLRS | |
| 15 | VDTYDGDISVV | |
| 16 | DTYDGDISVVY | |
| 17 | TYDGDISVVYG | |
| 18 | YDGDISVVYGL | |
| 19 | DGDISVVYGLR | FOL-062 |
| 20 | GDISVVYGLRS | |
| 21 | VDTYDGDISV | |
| 22 | DTYDGDISVV | |
| 23 | TYDGDISVVY | |
| 24 | YDGDISVVYG | |
| 25 | DGDISVVYGL | |
| 26 | GDISVVYGLR | FOL-009h |
| 27 | DISVVYGLRS | |
| 28 | VDTYDGDIS | FOL-019h |
| 29 | DTYDGDISV | |
| 30 | TYDGDISVV | |
| 31 | YDGDISVVY | |
| 32 | DGDISVVYG | |
| 33 | GDISVVYGL | |
| 34 | DISVVYGLR | |
| 35 | ISVVYGLRS | |
| 36 | VDTYDGDI | |
| 37 | DTYDGDIS | |
| 38 | TYDGDISV | |
| 39 | YDGDISVV | |
| 40 | DGDISVVY | |
| 41 | GDISVVYG | |
| 42 | DISVVYGL | |
| 43 | ISVVYGLR | |
| 44 | VDTYDGD | |
| 45 | DTYDGDI | |
| 46 | TYDGDIS | |
| 47 | YDGDISV | |
| 48 | DGDISVV | |
| 49 | GDISVVY | |
| 50 | DISVVYG | |
| 51 | ISVVYGL | |
| 52 | DTYDGD | |
| 53 | TYDGDI | |
| 54 | YDGDIS | |
| 55 | DGDISV | |
| 56 | GDISVV | |
| 57 | DISVVY | |
| 58 | ISVVYG | |
| 59 | TYDGD | |
| 60 | YDGDI | |
| 61 | DGDIS | |
| 62 | GDISV | |
| 63 | DISVV | |
| 64 | ISVVY | |
| 65 | SVVYG | |
| 66 | MRIAVICFCLLGITCAIPVKQADSGSSEEK | Wildtype |
| | QLYNKYPDAVATWLNPDPSQKQNLLAPQTL | human |
| | PSKSNESHDHMDDMDDEDDDDHVDSQDSID | osteopontin, |
| | SNDSDDVDDTDDSHQSDESHHSDESDELVT | i.e. |
| | DFPTDLPATEVFTPVVPTVDTYDGRGDSVV | GenBank: |
| | YGLRSKSKKFRRPDIQYPDATDEDITSHME | AAA59974.1 |
| | SEELNGAYKAIPVAQDLNAPSDWDSRGKDS | |
| | YETSQLDDQSAETHSHKQSRLYKRKANDES | |
| | NEHSDVIDSQELSKVSREFHSHEFHSHEDM | |
| | LVVDPKSKEEDKHLKFRISHELDSASSEVN | |
| 67 | VDTYDGRGDSVVYGLR | FOL-002 |
| 68 | VDZ₃Z₄Z₅GZ₇Z₈SZ₁₀Z₁₁YGLR | Z₃ is T or |
| | | V; |
| | | Z₄ is Y or |
| | | P; |
| | | Z₅ is D or |
| | | N; |
| | | Z₇ is D or |
| | | G; |
| | | Z₈ is I or |
| | | G; |
| | | Z₁₀ is V or |
| | | L; |
| | | Z₁₁ is V or |
| | | A |
| 69 | VDVPNGDISLAYGLR | FOL-004 |
| 70 | DVPNGDISLAYGLRS | |
| 71 | VDVPNGDISLAYGL | FOL-016 |
| 72 | DVPNGDISLAYGLR | FOL-007 |
| 73 | VPNGDISLAYGLRS | |
| 74 | VDVPNGDISLAYG | FOL-017 |
| 75 | DVPNGDISLAYGL | |
| 76 | VPNGDISLAYGLR | |
| 77 | PNGDISLAYGLRS | |
| 78 | VDVPNGDISLAY | |
| 79 | DVPNGDISLAYG | |
| 80 | VPNGDISLAYGL | |
| 81 | PNGDISLAYGLR | FOL-008 |
| 82 | NGDISLAYGLRS | |
| 83 | VDVPNGDISLA | FOL-018 |
| 84 | DVPNGDISLAY | |
| 85 | VPNGDISLAYG | |
| 86 | PNGDISLAYGL | |
| 87 | NGDISLAYGLR | |
| 88 | GDISLAYGLRS | |
| 89 | VDVPNGDISL | |
| 90 | DVPNGDISLA | |
| 91 | VPNGDISLAY | |
| 92 | PNGDISLAYG | |
| 93 | NGDISLAYGL | |
| 94 | GDISLAYGLR | FOL-009 |
| 95 | DISLAYGLRS | |
| 96 | VDVPNGDIS | FOL-019 |
| 97 | DVPNGDISL | |
| 98 | VPNGDISLA | |
| 99 | PNGDISLAY | |
| 100 | NGDISLAYG | |
| 101 | GDISLAYGL | |
| 102 | DISLAYGLR | |
| 103 | ISLAYGLRS | |
| 104 | VDVPNGDI | |
| 105 | DVPNGDIS | |
| 106 | VPNGDISL | |
| 107 | PNGDISLA | |
| 108 | NGDISLAY | |
| 109 | GDISLAYG | |
| 110 | DISLAYGL | |
| 111 | ISLAYGLR | |
| 112 | VDVPNGD | |
| 113 | DVPNGDI | |
| 114 | VPNGDIS | |
| 115 | PNGDISL | |
| 116 | NGDISLA | |
| 117 | GDISLAY | |
| 118 | DISLAYG | |
| 119 | ISLAYGL | |
| 120 | DVPNGD | |
| 121 | VPNGDI | |
| 122 | PNGDIS | |
| 123 | NGDISL | |
| 124 | GDISLA | |
| 125 | DISLAY | |
| 126 | ISLAYG | |
| 127 | VPNGD | |
| 128 | PNGDI | |
| 129 | NGDIS | |
| 130 | GDISL | |
| 131 | DISLA | |
| 132 | ISLAY | |
| 133 | SLAYG | |
| 134 | MRLAVICFCLFGIASSLPVKVTDSGSSEEK | Wildtype |
| | LYSLHPDPIATWLVPDPSQKQNLLAPQNAV | murine |
| | SSEEKDDFKQETLPSNSNESHDHMDDDDDD | osteopontin, |
| | DDDDGDHAESEDSVDSDESDESHHSDESDE | i.e. |
| | TVTASTQADTFTPIVPTVDVPNGRGDSLAY | NCBI |
| | GLRSKSRSFQVSDEQYPDATDEDLTSHMKS | Reference |
| | GESKESLDVIPVAQLLSMPSDQDNNGKGSH | Sequence: |
| | ESSQLDEPSLETHRLEHSKESQESADQSDV | NP_ |
| | IDSQASSKASLEHQSHKFHSHKDKLVLDPK | 001191162.1 |
| | SKEDDRYLKFRISHELESSSSEVN | |
| 135 | VDVPNGRGDSLAYGLR | FOL-001 |
| 136 | KPLAEIDSIELSYGIK | FOL-014 |
| 137 | GDPNDGRGDSVVYGLR | FOL-003 |
| 138 | VDTYDGGISVVYGLR | FOL-026 |
| 139 | VDTYDGDGSVVYGLR | FOL-027 |
| 140 | KX₂LAX₅X₆X₇X₈IX₁₀LX₁₂YGIK | X₂ is C, P |
| | | or G; |
| | | X₅ is E or |
| | | G; |
| | | X₆ is C, D |
| | | or I; |
| | | X₇ is D, I, |
| | | S or G; |
| | | X₈ is S, D |
| | | or G; |
| | | X₁₀ is E or |
| | | G; |
| | | X₁₂ is S or |
| | | T; |
| 141 | KCLAECDSIELSYGIK (Cyclic) | FOL-032 |
| 142 | CLAEIDSC (Cyclic) | FOL-033 |
| 143 | CFKPLAEIDSIECSYGIK (Cyclic) | FOL-036 |
| 144 | KPLAEDISIELSYGIK | FOL-037 |
| 145 | KPLAEISDIELSYGIK | FOL-038 |
| 146 | KPLAEIGDIELSYGIK | FOL-039 |
| 147 | KPLAEGDIELSYGIK | FOL-040 |
| 148 | KPLAEIELSYGIK | FOL-041 |
| 149 | KPLAEIDSIELTYGIK | FOL-042 |
| 150 | KPLAEIDGIELSYGIK | FOL-043 |
| 151 | KPLAEIDGIELTYGIK | FOL-044 |
| 152 | KPLAEIGSIELSYGIK | FOL-045 |
| 153 | KGLAEIDSIELSYGIK | FOL-046 |
| 154 | KPLAGIDSIGLSYGIK | FOL-047 |
| 155 | Cyclic KCLAEIDSCELSYGIK | FOL-034 |
| 156 | Cyclic CFKPLAEIDSIEC | FOL-035 |
| 157 | VDVPEGDISLAYGLR | FOL-010 |
| 158 | LDGLVRAYDNISPVG | FOL-015 |
| 159 | GDPNGDISVVYGLR | FOL-006 |
| 160 | VDVPNGDISLAYRLR | FOL-011 |
| 161 | VDVPEGDISLAYRLR | FOL-012 |
| 162 | KX₂LAX₅X₆X₇X₈IX₁₀LSYGIK | X₂ is C, P |
| | | or G; |
| | | X₅ is E or |
| | | G; |
| | | X₆ is C, I |
| | | or absent; |
| | | X₇ is D, G |
| | | or absent; |
| | | X₈ is S, G |
| | | or absent; |
| | | X₁₀ is E or |
| | | G; |
| 163 | KX₂LAX₅IX₁₀LSYGIK | X₂ iS C, P |
| | | or G; |
| | | X₅ is E or |
| | | G; |
| | | X₁₀ is E or |
| | | G. |
| 164 | VDVPZ₅GDISLAYZ₁₃LR | Z₅ is E or |
| | | N; |
| | | Z₁₃ is R or |
| | | G. |
| 165 | VDTYDGZ₇Z₅SVVYGLR | Z₇ is D or |
| | | G; |
| | | Z₈ is I or |
| | | G. |
| 166 | GDPNZ₅Z₆Z₇Z₅Z₉SVVYGLR | Z₅ is D or |
| | | G; |
| | | Z₆ is D or |
| | | G |
| | | Z₇ is I or |
| | | R; |
| | | Z₈ is G or |
| | | absent; |
| | | Z₉ is D or |
| | | absent. |
| 167 | VZ₂TYDGDISVVYGLR | Z₂ is beta |
| | | D |
| | | FOL-005 |
| | | (2betaAsp) |
| 168 | VDTYZ₅GDISVVYGLR | Z₅ is beta |
| | | D |
| | | FOL-005 |
| | | (5betaAsp) |
| 169 | VDTYDGZ₇ISVVYGLR | FOL-005 |
| | | (7betaAsp) |
| | | Z₇ is beta |
| | | D |
| 170 | LAEIDSIELSYGIK | |
| 171 | AEIDSIELSYGIK | FOL-056 |
| 172 | EIDSIELSYGIK | FOL-057 |
| 173 | IDSIELSYGIK | FOL-058 |
| 174 | DSIELSYGIK | FOL-059 |
| 175 | SIELSYGIK | FOL-060 |
| 176 | IELSYGIK | |
| 177 | X₁LX₂YGIK | X₁ is E or |
| | | G; |
| | | X₂ is S or |
| | | T; |
| 178 | Z₁Z₂SZ₃Z₄YGLR | Z₁ is D or |
| | | G; |
| | | Z₂ is I or |
| | | G; |
| | | Z₃ iS V or |
| | | L; |
| | | Z₄ is V or |
| | | A. |
| 179 | KPLAEIDSIELSYGI | |
| 180 | KPLAEIDSIELSYG | |
| 181 | KPLAEIDSIELSY | |
| 182 | KPLAEIDSIELS | |
| 183 | KPLAEIDSIEL | |
| 184 | KPLAEIDSIE | |

The invention is further illustrated by the following two examples, which however should not be construed as being limiting for the invention. These examples demonstrate that exemplary peptide of the present invention and have the ability to stimulate insulin secretion from rat β-cells and to protect β-cells from the effects of glucotoxic conditions, by retaining their capacity to secrete insulin.

### Example 18: FOL-056 Induce Insulin Secretion from INS-1E cells

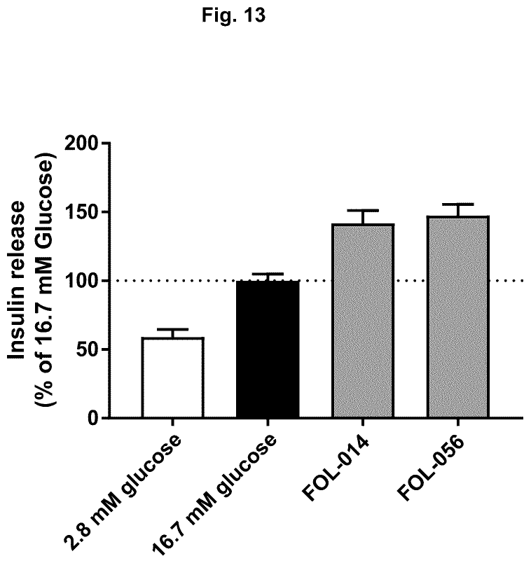

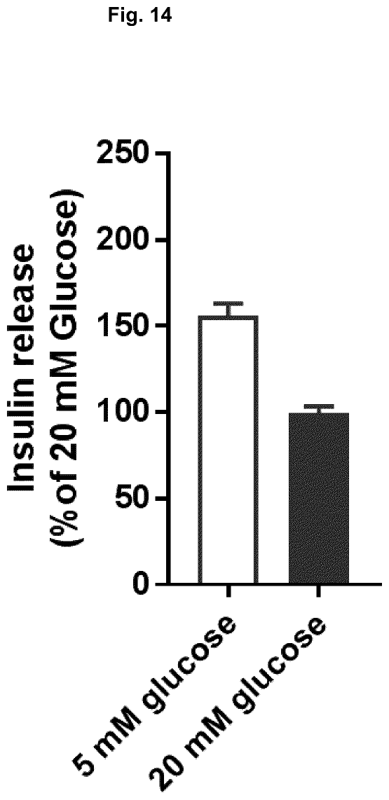

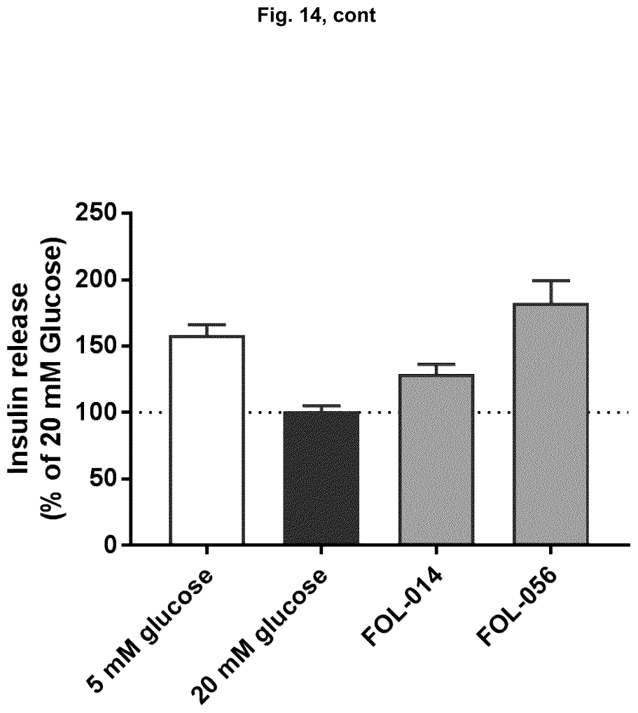

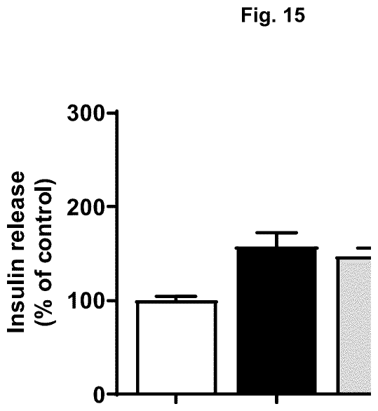

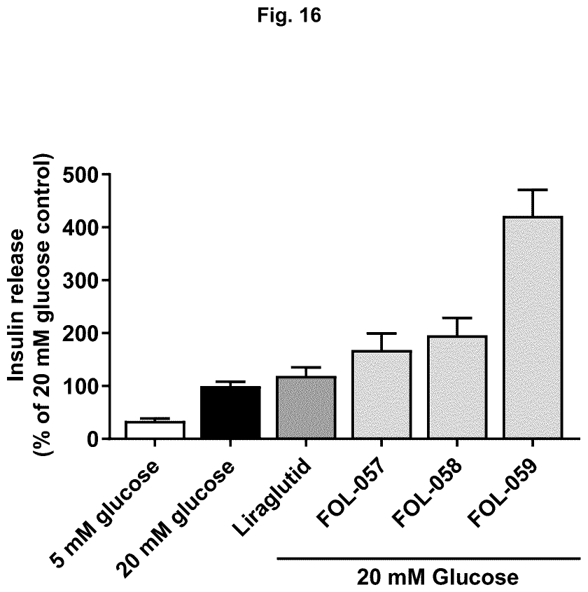

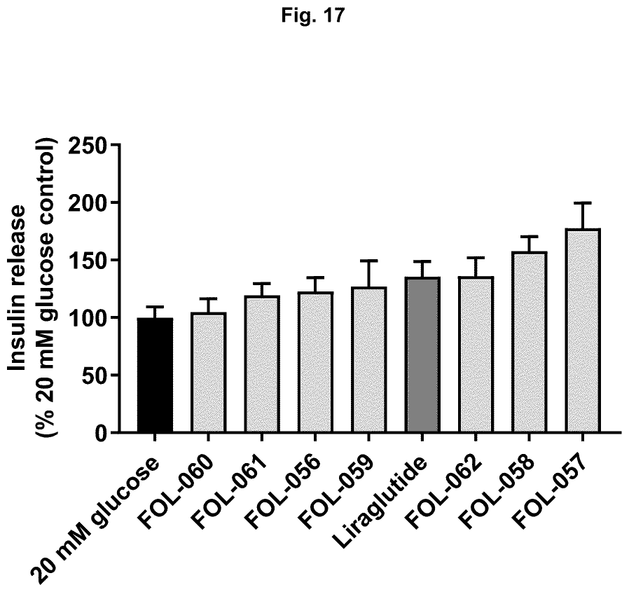

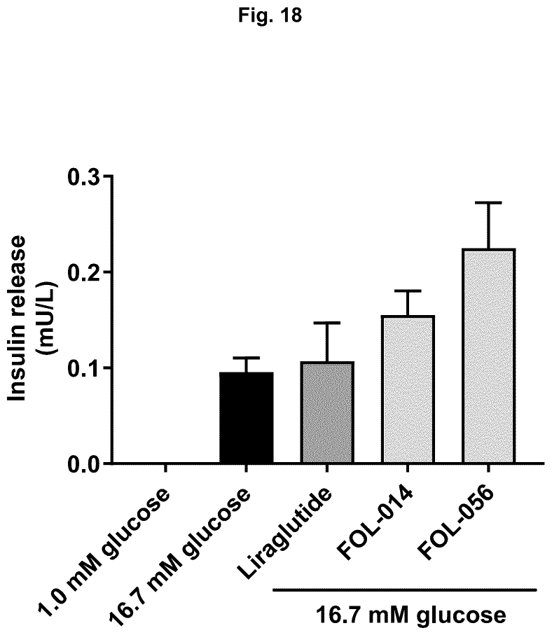

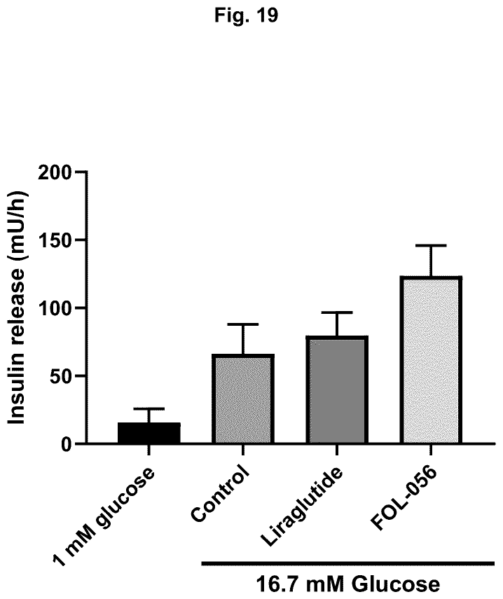

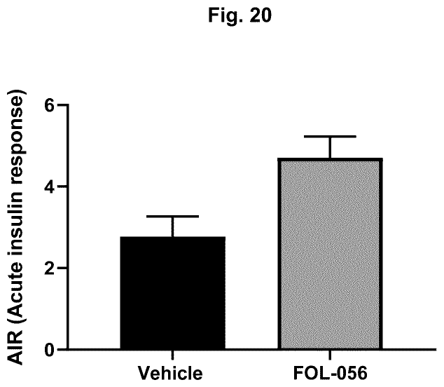

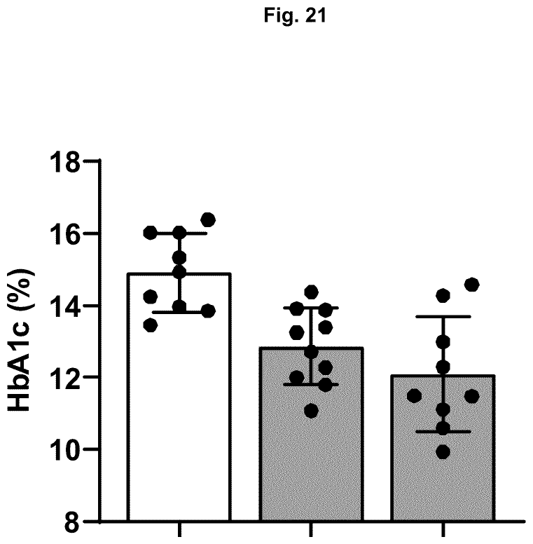

To investigate the stimulatory effect of FOL-056 on insulin secretion we used INS-1E cells. The cells were seeded overnight and washed with PBS before pre-incubation for 60 min at 37° C. in Krebs-Ringer bicarbonate buffer (KRB), pH 7.4, supplemented with 10 mM HEPES, 0.1% bovine serum albumin. Following pre-incubation, the buffer was discarded and the INS-1E cells were incubated in fresh KRB buffer supplemented with 10 mM HEPES, 0.1% bovine serum albumin with or without peptide FOL-056. For comparative purposes cells treated with FOL-014 was included. Following 60 min incubation at 37.C, the buffer was removed and frozen for subsequent insulin assay. The results demonstrate that β-cells stimulated with the peptide FOL-056 secrete significantly more insulin compared to unstimulated control cells (FIG. 13).

### Example 19: FOL-056 Preserves the Insulin Secreting Capacity of INS-1E Cells During Long-Term Glucotoxic Conditions

To investigate the β-cell protective effect of FOL-056, we subjected INS-1E to cytotoxic levels of glucose for 72 hours. Rat INS-1E cells were seeded in 96-well plates (2×103 cells/well) in cRPMI medium. Following 72 hours of incubation, the medium was changed to RPMI containing 20 mM glucose with or without FOL-056 or FOL-014 and were cultured at 37° C. during an additional 72 hours to induce glucotoxicity. RPMI containing 5 mM glucose was included as a low glucose control. Following 72 hours, the medium was removed and the INS-1E cells were equilibrated in Krebs-Ringer bicarbonate buffer (KREB), pH 7.4, (supplemented with 10 mM HEPES, 0.1% bovine serum albumin and 2.8 mM glucose) for 2 hours. After equilibration, the buffer was changed and the INS-1E cells were incubated in KREBs containing 16.7 mM glucose supplemented with during 1 h. Immediately after incubation, an aliquot of the buffer was removed and frozen for subsequent assay of insulin content.

The results demonstrate that the presence FOL-056 during glucotoxic conditions preserves β-cell function as shown by retained glucose induced insulin secretion.

### Example 20. FOL-056 in Combination with GLP-1 Increases Insulin Secretion as Compared with Either Peptide Alone

Insulin secretion from INS-1 cells was measured following exposure to FOL-056, GLP-1 or a combination of those, expressed as percentage of untreated control. The combined effect of GLP-1 and FOL-056 resulted in a significantly higher insulin release than GLP-1 or FOL-056 alone. The experiments were performed in the presence of 16.7 mM glucose (FIG. 15).

The results demonstrate that the combination of GLP-1 and fragments of FOL-peptides further potentiate the insulin secretion from INS-1 cells in vitro as compared with each peptide alone.

### Example 21. Novel Peptide Analogues Induce Insulin Secretion in Pancreatic β-Cell-Lines

Novel peptide analogues, were tested to investigate their ability to induce insulin secretion in an INS-1 cell line in the presence of 20 mM glucose. Liraglutide as well as a high glucose (20 mM) and a low glucose (5 mM) control were included in each experiment (peptide concentration was 100 nM). In order to correct for the variance between experiments, all values were normalized to, and expressed as percentage of the average value of the high glucose control in the individual experiments. The analogues were subsequently ranked according to performance (FIG. 16).

The results demonstrate the capacity of the novel peptide analogues to enhance insulin secretion from INS-1 β-cells in vitro.

### Example 22: Novel Peptides Derived from FOL-005 and FOL-014 Preserve the Insulin Secreting Capacity of INS-1 Cells During Long-Term Glucotoxic Conditions

To investigate the β-cell protective effect of several novel peptide fragments derived from FOL-005 and FOL-014, INS-1 cells were subjected to cytotoxic levels of glucose for 72 hours. The rat INS-1 cells were seeded in 96-well plates (2×10³ cells/well) in CRPMI medium. Following 72 hours of incubation, the medium was changed to RPMI containing 20 mM glucose with or without peptides and the cells were cultured at 37° C. during an additional 72 hours to induce glucotoxicity. RPMI containing 5 mM glucose was included as a low glucose control (not shown) and liraglutide was included for comparison. Following 72 hours, the medium was removed and the INS-1 cells were equilibrated in Krebs-Ringer bicarbonate buffer (KREB), pH 7.4, (supplemented with 10 mM HEPES, 0.1% bovine serum albumin and 2.8 mM glucose) for 2 hours. After equilibration, the buffer was changed and the INS-1 cells were incubated in KREBs containing 16.7 mM glucose supplemented with during 1 h. Immediately after incubation, an aliquot of the buffer was removed and frozen for subsequent assay of insulin content. In order to correct for the variance between experiments, all values were normalized to, and expressed as percentage of the average value of the high glucose control in the individual experiments. The analogues were subsequently ranked according to performance (FIG. 17).

The results demonstrate that the presence of the several novel peptides during glucotoxic conditions preserves β-cell function as shown by retained glucose induced insulin secretion.

### Example 23: Stimulation of Insulin Secretion from Human Pancreatic β-Cells by FOL-056 Peptides

In order to test the effect of FOL-014 and FOL-056 in human cells, the human pancreatic β-cell line 1.2B4 were cultured at 37° C. and 5% CO₂ in RPMI 1640 supplemented with 10% fetal bovine serum, 50 IU/mL penicillin, 50 mg/L streptomycin, 1 mM L-glutamine. 1.2B4 cells were seeded in 24-well plates in RPMI medium and following overnight incubation, the medium was removed before pre-incubation for 40 min at 37° C. in Krebs-Ringer bicarbonate buffer, pH 7.4, supplemented with 10 mM HEPES, 0.1% bovine serum albumin and 1.0 mM glucose. Following pre-incubation, the buffer was exchanged with fresh Krebs-Ringer buffer as described above and supplemented with specific glucose concentrations, 1 mM or 16.7 mM. FOL-014, FOL-056 or Liraglutide was added at a concentration of 100 nM in the presence of 16.7 mM glucose. Immediately after 60 minutes incubation at 37° C., an aliquot of the buffer was removed and frozen for subsequent insulin ELISA assay. (FIG. 18)

The results demonstrate that the peptides FOL-014 and FOL-056 increase the insulin secretion capacity in 1.2B4 human β-cells in the presence of 16.7 mM glucose.

### Example 24: Stimulation of Insulin Secretion from Human Pancreatic Islets by FOL-056 Peptides

To investigate the functionality of FOL-056 in human primary tissue, the pancreatic islets from two non-diabetic human donors were used. Islets were picked, aliquoted in groups of 12 and incubated at 37° C. in 1 ml Krebs-Ringer bicarbonate buffer, pH 7.4, supplemented with 10 mM HEPES, 0.1% bovine serum albumin and 1.0 mM glucose. Following pre-incubation, the buffer was exchanged with fresh Krebs-Ringer buffer as described above and supplemented with specific glucose concentrations (1 mM or 16.7 mM), FOL-056 peptides (1 nM) or Liraglutide (100 nM). Immediately after 60 minutes incubation at 37° C., an aliquot of the buffer was removed and frozen for subsequent insulin ELISA assay. (FIG. 19)

The results demonstrate that FOL-056 potentiates the capacity of primary human pancreatic islets to secrete insulin as a response to high glucose levels.

### Example 25 and 26: Long Term Dosing of Diet Induces Obese c57B16 Mice

Materials and methods: To investigate the effects of FOL-056 in vivo in a high fat diet model, wild type c57BI6 mice were subcutaneously dosed 5 days per week with 300 nmol/kg FOL-056, while being fed high fat diet for 12 weeks. Control animals were dosed with PBS.

### Example 25: Long Term Dosing with FOL-056 Increases the Acute Insulin Response In Vivo

In diet induced obese c57BI6 mice, untreated or dosed with FOL-056, the fasting plasma insulin levels were measured before as well as 1 minute after an intravenous glucose injection of 1 g/kg. The insulin value measured before the glucose injection was subtracted from the value measured after the injection for each individual mouse in order to obtain the acute insulin response (AIR). (FIG. 20)

The results demonstrate that the acute insulin response (AIR) was significantly improved in mice treated with FOL-056 as compared with untreated control mice.

### Example 26: Reduction of HbA1c in Diabetic Mice Following 4 Weeks of Dosing

To investigate the long-term effect of FOL-014 and FOL-056 in diabetic mice, db/db mice were dosed with 100 nmol/kg peptide subcutaneously, 5 days per week for 4 weeks. Control mice were injected with PBS. After 4 weeks of treatment the mice were terminated and 25 μl whole blood was immediately frozen for subsequent HbA1c analysis. (FIG. 21).

The results demonstrate that 4 weeks of treatment with FOL-014 and FOL-056 reduce the HbA1c in db/db mice as compared with the untreated control group.

1. A peptide comprising the amino acid sequence IELSYGIK (SEQ ID NO: 176), with the proviso that the peptide comprises no more than 85 amino acid residues, and with the proviso that the peptide is not KPLAEIDSIELSYGIK (SEQ ID NO: 136), KCLAECDSIELSYGIK (SEQ ID NO: 141), KPLAEDISIELSYGIK (SEQ ID NO: 144), KPLAEISDIELSYGIK (SEQ ID NO: 145), KPLAEIGDIELSYGIK (SEQ ID NO: 146), KPLAEGDIELSYGIK (SEQ ID NO: 147), KPLAEIELSYGIK (SEQ ID NO: 148), KPLAEIDGIELSYGIK (SEQ ID NO: 150), KPLAEIGSIELSYGIK (SEQ ID NO: 152), or KGLAEIDSIELSYGIK (SEQ ID NO: 153).

2. The peptide according to claim 1, wherein the peptide comprises no more than 80, such as no more than 75, such as no more than 70, such as no more than 65, such as no more than 60, such as nor more than 55, such as no more than 50, such as no more than 55, such as no more than 40 amino acids, such as no more than 35, such as no more than 30, such as no more than 28, such as no more than 26, such as no more than 24, such as no more than 22, such as no more than 20, such as no more than 19, such as no more than 18, such as no more than 17, such as no more than 16, such as no more than 15, such as no more than 14, such as no more than 13, such as no more than 12, such as no more than 11, such as no more than 10 amino acids.

3. The peptide according to claim 1, wherein the peptide comprises an amino acid sequence selected from the group consisting of AEIDSIELSYGIK (SEQ ID NO: 171), SIELSYGIK (SEQ ID NO: 175), DSIELSYGIK (SEQ ID NO: 174), IDSIELSYGIK (SEQ ID NO: 173), EIDSIELSYGIK (SEQ ID NO: 172), and LAEIDSIELSYGIK (SEQ ID NO: 170).

4. The peptide according to claim 1, wherein the peptide consists of the amino acid sequence AEIDSIELSYGIK (SEQ ID NO: 171).

5. The peptide according to claim 1, wherein the peptide consists of the amino acid sequence LAEIDSIELSYGIK (SEQ ID NO: 170).

6. The peptide according to claim 1, wherein the peptide consists of the amino acid sequence EIDSIELSYGIK (SEQ ID NO: 172).

7. The peptide according to claim 1, wherein the peptide consists of the amino acid sequence IDSIELSYGIK (SEQ ID NO: 173).

8. The peptide according to claim 1, wherein the peptide consists of the amino acid sequence DSIELSYGIK (SEQ ID NO: 174).

9. The peptide according to claim 1, wherein the peptide consists of the amino acid sequence SIELSYGIK (SEQ ID NO: 175).

10. The peptide according to claim 1, wherein the peptide consists of the amino acid sequence IELSYGIK (SEQ ID NO: 176).

11. The peptide according to any one of the preceding claims, wherein the peptide is conjugated to a moiety.

12. The peptide according to claim 11, wherein the moiety is selected from the group consisting of polyethylene glycol (PEG), monosaccharides, fluorophores, chromophores, radioactive compounds, and cell-penetrating peptides.

13. The peptide according to any one of the preceding claims, wherein the peptide is further modified, such as being glycosylated or by PEGylation, amidation, esterification, acylation, acetylation and/or alkylation.

14. The peptide according to any one of the preceding claims, wherein the peptide comprises or consists of tandem repeats.

15. The peptide according to claim 14, wherein the tandem repeats comprise or consist of the amino acid sequence of any one or more of the sequences as described in the preceding claims.

16. The peptide according to any one of the preceding claims, wherein the peptide is fused to another polypeptide.

17. The peptide according to claim 16, wherein the polypeptide is selected from the group consisting of glutathione-S-transferase (GST) and protein A.

18. The peptide according to any of the preceding claims, wherein the peptide is fused to a tag.

19. The peptide according to claim 18, wherein the tag is an oligo-histidine tag.

20. The peptide according to any one of the preceding claims, wherein the peptide is cyclic.

21. The peptide according to any of the preceding claims, wherein the peptide is capable of forming at least one intramolecular cysteine bridge.

22. The peptide according to any one of the preceding claims, wherein the peptide comprises an amino acid residue P at the N-terminus.

23. The peptide according to any one of the preceding claims, wherein the peptide has 1 additional amino acid.

24. The peptide according to any one of the preceding claims, wherein the peptide further comprises a detectable moiety.

25. The peptide according to any one of the preceding claims, wherein the detectable moiety comprises or consists of a radioisotope.

26. The peptide according to any one of the preceding claims, wherein the radioisotope is selected from the group consisting of ^{99m}Tc, ¹¹¹In, ⁶⁷Ga, ⁶⁸Ga, ⁷²As, ⁸⁹Zr, ¹²³I and ²⁰¹TI.

27. The peptide according to any one of the preceding claims, wherein the detectable moiety is detectable by an imaging technique such as SPECT, PET, MRI, optical or ultrasound imaging.

28. Use of the peptide according to any of the preceding claims, for the preparation of a diagnostic composition for the diagnosis of a disease, disorder or damage of the pancreas in an individual.

29. A polynucleotide encoding upon expression, a peptide according to any one of claims 1 to 27.

30. A vector comprising the polynucleotide according to claim 29.

31. A cell comprising the polynucleotide according to claim 29, or the vector according to claim 30.

32. A composition comprising a peptide according to any one of claims 1 to 27, a polynucleotide according to claim 29, a vector according to claim 30 or a cell according to claim 31.

33. The composition according to claim 32, wherein the composition is a pharmaceutical composition.

34. The peptide according to any one of claims 1 to 27, the polynucleotide according to claim 29, the vector according to claim 30, the cell according to claim 31 or the composition according to any one of claim 32 or 33, for use as a medicament.

35. The peptide according to any one of claims 1 to 27, the polynucleotide according to claim 29, the vector according to claim 30, the cell according to claim 31 or the composition according to any one of claim 32 or 33 for use in the treatment of an endocrine disease, a nutritional disease and/or a metabolic disease in a mammal.

36. The peptide or the composition for use according to any one of the preceding claims, wherein said peptide or composition comprises a second or further active ingredient.

37. The peptide or the composition for use according to claim 36, wherein the second or further active ingredient is selected from the group consisting of insulin, glucagon-like peptide-1 (GLP-1), sulfonylurea, a dipeptidyl peptidase-4 (DPP4) inhibitor, an alpha-glucosidase inhibitor, a thiazolidinedione, a meglitinide and a sodium-glucose cotransporter-2 (SGLT2) inhibitor.

38. The peptide according to any one of claims 1 to 27, the polynucleotide according to claim 29, the vector according to claim 30, the cell according to claim 31 or the composition according to any one of claim 32 or 33 for use according to claim 35, wherein the mammal is a human.

39. The peptide according to any one of claims 1 to 27, the polynucleotide according to claim 29, the vector according to claim 30, the cell according to claim 31 or the composition according to any one of claim 32 or 33 for use according to claim 35, wherein the endocrine disease, nutritional disease and/or metabolic disease are selected from the group consisting of diabetes mellitus, type 1 diabetes mellitus, type 2 diabetes mellitus, malnutrition-related diabetes mellitus, disorders of glucose regulation and pancreatic internal secretion, insulin resistance syndrome, impaired glucose tolerance, hyperglycemia, hyperinsulinemia, and any combinations thereof.

40. The peptide according to any one of claims 1 to 27, the polynucleotide according to claim 29, the vector according to claim 30, the cell according to claim 31 or the composition according to any one of claim 32 or 33 for use according to claim 39, wherein the diabetes mellitus is selected from the group consisting of type 1 diabetes mellitus, type 2 diabetes mellitus, malnutrition-related diabetes mellitus, specified diabetes mellitus, and unspecified diabetes mellitus.

41. A method of treating an endocrine disease, a nutritional disease and/or a metabolic disease, the method comprising administering the peptide according to any one of claims 1 to 27, the polynucleotide according to claim 29, the vector according to claim 30, the cell according to claim 31 or the composition according to any one of claim 32 or 33, to a subject in need thereof.

42. Use of the peptide according to any one of claims 1 to 27, the polynucleotide according to claim 29, the vector according to claim 30, the cell according to claim 31 or the composition according to any one of claim 32 or 33 for the manufacture of a medicament for the treatment of an endocrine disease, a nutritional disease and/or a metabolic disease in a mammal.

43. A method for delaying onset of diabetes and/or a diabetes associated disorder or disease, the method comprising administering a therapeutically effective amount of the peptide according to any one of claims 1 to 27, the polynucleotide according to claim 29, the vector according to claim 30, the cell according to claim 31 or the composition according to any one of claim 32 or 33, to an individual in need thereof.

44. A method for decreasing blood glucose levels, the method comprising administering a therapeutically effective amount of the peptide according to any one of claims 1 to 27, the polynucleotide according to claim 29, the vector according to claim 30, the cell according to claim 31 or the composition according to any one of claim 32 or 33, to an individual in need thereof.

45. The method according to claim 44, wherein insulin secretion is increased.

46. The method according to claim 44, wherein cellular uptake of glucose is increased.

47. The method according to claim 44, wherein the insulin production is increased.

48. The method according to claim 44, wherein the glucagon production is decreased.

49. A method for improving beta cell viability, the method comprising administering a therapeutically effective amount of the peptide according to any one of claims 1 to 27, the polynucleotide according to claim 29, the vector according to claim 30, the cell according to claim 31 or the composition according to any one of claim 32 or 33, to an individual in need thereof.

50. A method for improving beta cell morphology, the method comprising administering a therapeutically effective amount of the peptide according to any one of claims 1 to 27, the polynucleotide according to claim 29, the vector according to claim 30, the cell according to claim 31 or the composition according to any one of claim 32 or 33, to an individual in need thereof.

51. A method for stabilising or improving viability and/or morphology of pancreatic islets, the method comprising administering a therapeutically effective amount of the peptide according to any one of claims 1 to 27, the polynucleotide according to claim 29, the vector according to claim 30, the cell according to claim 31 or the composition according to any one of claim 32 or 33, to an individual in need thereof.
